(19) United States
(12) Patent Application Publication
(10) Pub. No.: US 2003/0069629 A1
(43) Pub. Date: Apr. 10, 2003

(54) Bioresorbable medical devices

(76) Inventors: Balkrishna S. Jadhav, Plymouth, MN (US); Robert C. Grant, New Hope, MN (US)

Correspondence Address: OPPENHEIMER WOLFF & DONNELLY LLP, 840 NEWPORT CENTER DRIVE, SUITE 700, NEWPORT BEACH, CA, 92660, US

(21) Appl. No.: 10/128,867

(22) Filed: Apr. 23, 2002

Related U.S. Application Data
(63) Continuation-in-part of application No. 09/920,871, filed on Aug. 2, 2001.
(60) Provisional application No. 60/295,327, filed on Jun. 1, 2001.
(60) Provisional application No. 60/304,592, filed on Jul. 9, 2001.

(51) Int. Cl. A61F 2/06

(52) U.S. Cl. 623/1.15

(57) ABSTRACT

A bio-compatible and bioresorbable medical device is disclosed. Specifically a polymeric stent is disclosed intended to restore or maintain patency following surgical procedures, traumatic injury or stricture formation. The polymeric stent is composed of one or more polymers that is either extruded as a monofilament then woven into a braid-like embodiment, or injection molded or extruded as a tube with fenestrations in the wall. Related methods for controlling the medical devices' in vivo functional life by controlling polymer monomer content and other polymer structural qualities are also provided.

### RELATED APPLICATIONS

This application is a continuation-in-part of co-pending U.S. patent application Ser. Nos. 09/920,871 filed Aug. 2, 2001 and provisional application serial Nos. 60/295,327 filed Jun. 1, 2001 and 60/304,592 filed Jul. 9, 2001. The entire contents of which are herein incorporated by reference.

### FIELD OF THE INVENTION

This invention relates to implantable medical devices, and particularly to bioresorbable, biocompatible medical devices. Specifically, biocompatible, bioresorbable stents useful in the treatment of strictures and preventing restenosis.

### BACKGROUND

Tubular organs and structures such as blood vessels, the esophagus, intestines, endocrine gland ducts and the urethra are all subject to strictures, i.e., a narrowing or occlusion of the lumen. Strictures can be caused by a variety of traumatic or organic disorders and symptoms can range from mild irritation and discomfort to paralysis and death. Treatment is site specific and varies with the nature and extent of the occlusion.

Life threatening stenoses are most commonly associated with the cardiovascular system and are often treated using percutaneous transluminal coronary angioplasty (PTCA). This process reduces the stricture by expanding the artery's diameter at the blockage site using a balloon catheter. However, three to six months after PTCA, approximately 30% to 40% of patients experience restenosis. Injury to the arterial wall during PTCA is believed to be the initiating event causing restenosis and primarily results from vascular smooth muscle cell proliferation and extracellular matrix secretion at the injured site. Restenosis is also a major problem in non-coronary artery disease including the carotid, femoral, iliac, popliteal and renal arteries.

Stenosis of non-vascular tubular structures is often caused by inflammation, neoplasm and benign intimal hyperplasia. In the case of esophageal and intestinal strictures, the obstruction can be surgically removed and the lumen repaired by anastomosis. The smaller transluminal spaces associated with ducts and vessels may also be repaired in this fashion; however, restenosis caused by intimal hyperplasia is common. Furthermore, dehiscence is also frequently associated with anastomosis requiring additional surgery which can result in increased tissue damage, inflammation and scar tissue development leading to restenosis.

Problems with diminished urine flow rates are common in aging males. The most frequent cause is benign prostatic hypertrophy (BPH). In this disease the internal lobes of the prostate slowly enlarge and progressively occlude the urethral lumen. A number of therapeutic options are available for treating BPH. These include watchful waiting (no treatment), several drugs, a variety of so-called “less invasive” therapies, and transurethral resection of the prostate (TURP)—long considered the gold standard.

Urethral strictures are also a significant cause of reduced urine flow rates. In general, a urethral stricture is a circumferential band of fibrous scar tissue which progressively contracts and narrows the urethral lumen. Strictures of this type may be congenital or may result from urethral trauma or disease. Strictures were traditionally treated by dilation with sounds or bougies. More recently, balloon catheters became available for dilation. Surgical urethrotomy is currently the preferred treatment, but restenosis remains a significant problem.

Recent advances in biomedical engineering have led to the development of stenting, i.e., mechanical scaffolding, to prevent restenosis and keep the previously occluded lumens open. There are two general types of stents: permanent and temporary. Temporary stents can be further subdivided into removable and absorbable.

Permanent stents are used where long term structural support or restenosis prevention is required, or in cases where surgical removal of the implanted stent is impractical. Permanent stents are usually made from metals such as Phynox, 316 stainless steel, MP35N alloy, and superelastic Nitinol (nickel-titanium).

Stents are also used as temporary devices to prevent closure of a recently opened urethra following minimally invasive procedures for BPH which typically elicit post treatment edema and urethral obstruction. In these cases, the stent will typically not be covered with tissue (epithelialized) prior to removal.

Temporary absorbable stents can be made from a wide range of synthetic bio-compatible polymers depending on the physical qualities desired. Representative bio-compatible polymers include polyanhydrides, polycaprolactone, polyglycolic acid, poly-L-lactic acid, poly-D-L-lactic acid and polyphosphate esters.

Stents are designed to be deployed and expanded in different ways. A stent can be designed to self expand upon release from its delivery system, or it may require application of a radial force through the delivery system to expand the stent to the desired diameter. Self expanding stents are typically made of metal and are woven or wound like a spring. Synthetic polymer stents of this type are also known in the art. Self-expanding stents are compressed prior to insertion into the delivery device and released by the practitioner when correctly positioned within the stricture site. After release, the stent self expands to a predetermined diameter and is held in place by the expansion force or other physical features of the device.

Stents which require mechanical expansion by the surgeon are commonly deployed by a balloon-type catheter. Once positioned within the stricture, the stent is expanded in situ to a size sufficient to fill the lumen and prevent restenosis. Various designs and other means of expansion have also been developed. One variation is described in Healy and Dorfman, U.S. Pat. No. 5,670,161. Healy and Dorfman disclose the use of a bio-compatible stent that is expanded by a thermo-mechanical process concomitant with deployment.

Approximately one-third of all patients undergoing surgery, catheterization or balloon dilation to repair bulbar urethral strictures experience restenosis. In these patients the use of urethral stents has provided satisfactory relief from symptoms. (Badlani, G. H., et al., UroLume® Endourethral Prosthesis for the Treatment of Urethral Stricture Disease: Long-term Results of the North American Multicenter UroLume® Trail. Urology: 45:5, 1993). Currently, urethral stents are composed of bio-compatible metals woven into a tubular mesh or wound into a continuous coil and are inserted endoscopically after opening the stricture by urethrotomy or sequential dilation. The stent is initially anchored in place through radial force as the stent exerts expansion pressure against the urethral wall. With woven stents epithelial cells lining the urethra begin to grow through the stent's open weave between six and 12 weeks after insertion, thereby permanently securing the stent.

For most patients this is a one time process without complication. However, some men experience post insertion complications including stent migration, excessive epithelialization, and stent encrustation. In some cases excessive epithelial tissue may be resected transurethrally. In other situations stent removal may be necessary. Depending on the condition of the stent, removal procedures range from a relatively simple transurethral procedure to open surgery with excision and urethroplasty. All complications increase patent discomfort and health care costs.

Recently, a number of bio-compatible, bioresorbable materials have been used in stent development and in situ drug delivery development. Examples include U.S. Pat. Nos. 5,670,161 (a thermo-mechanically expanded biodegradable stent made from a co-polymer of L-lactide and ε-caprolactone), 5,085,629 (a bioresorbable urethral: stent comprising a terpolymer of L-lactide, glycolide and ε-caprolactone) 5,160,341 (a resorbable urethral stent made from polylactic acid or polyglycolic acid), and 5,441,515 (a bio-erodible drug delivery stent and method with a drug release layer). These bioresorbable stents gradually hydrolyze in the body and stent fragments are then excreted, as in the case of urethral and bowel stents, or the nontoxic soluble degradation products may be absorbed and metabolized. Consequently, the use of bioresorbable stents may ultimately eliminate the need for invasive removal procedures.

However, advancements in polymeric, bio-resorbable stent design is still needed. Given, for example, there remains a need for bioresorbable stents that provide enough radial strength to maintain luminal patency over a wide range of medical conditions and implantation sites. Furthermore, there is also a need to have bioresorbable stents that have controlled degradation without total stent collapse and resulting obstruction. Moreover, there is a need for cost-effective biocompatible stents and processes for making stents that have differing functional lives.

### SUMMARY OF THE INVENTION

The present invention relates to implantable, bioresorbable, biocompatible polymeric medical devices and methods for making same. Moreover, the implantable, bioresorbable, biocompatible polymeric medical devices of the present invention are intended for short to medium term in vivo use. The biocompatible, bioresorbable medical devices of the present invention can be made from a variety of biocompatible polymeric compounds, their respective monomers, dimers, oligomers and blends thereof. For example, and not intended as a limitation, the polymers used to make present invention include polyanhydrides, polycaprolactones, polyglycolic acids, poly-L-lactic acids, poly-D-L-lactic acids, and polyphosphate esters and their respective monomers, dimers, and oligomers. The polymeric materials of the present invention can be formed using techniques known to those having ordinary skill in the art of polymer chemistry and the material sciences. The polymers can be extruded into monofilaments, sheets or tubes and other configurations.

It is an object of the present invention to provide medical devices that will temporarily restore, or maintain patency of tubular anatomical structures such as, but not limited to, blood vessels, the bile duct, the ureter, the urethra, and the intestines. It is another objective of the invention to provide biocompatible medical devices that are bioresorbable, thus eliminating the need for costly, painful and potentially damaging post insertion removal.

In one embodiment of the present invention, the medical device is a biological stent, specifically a urethral stent. In another embodiment of the present invention, the medical device is a stent woven from a plurality of extruded polymeric monofilaments. In another embodiment the stent is extruded or injection molded as a tubular structure having fenestrations therein or provided with fenestrations thereafter using techniques known to those having ordinary skill in the art.

Another embodiment of the present invention includes bioresorbable stents having a radially self-expanding, tubular shaped member which may also expand and contract along its horizontal axis (axially retractable). The stent having first and second ends and a walled surface disposed between the first and second ends. The walled surface may include a plurality of substantially parallel pairs of monofilaments with the substantially parallel pairs of monofilaments woven in a helical shape. The stent is woven such that one-half of the substantially parallel pairs of monofilaments are wound clockwise in the longitudinal direction and one-half of the substantially parallel pairs of monofilaments are wound counterclockwise in the longitudinal direction. This results in a stent having an alternating, over-under plait of the oppositely wound pairs of monofilaments.

Still another embodiment of the present invention may include a radially expandable, axially retractable bioresorbable stent made from biocompatible, bioresorbable polymers injection molded into a substantially tubular shaped device. The injection molded or extruded tubular shape device may have first and second ends with a walled structure disposed between the first and second ends and wherein the walled structure has fenestrations therein.

In yet another embodiment of the present invention, the in vivo functional life of the stent is adjusted using methods comprising post-stent formation treatment steps selected from the group consisting of annealing gamma irradiation and combinations thereof.

In another embodiment of the present invention, the monomer content in the polymeric material is adjusted prior to stent formation using polymer extrusion pressure.

In another embodiment of the present invention, the monomeric content is adjusted in the polymeric material using processes of blending polymeric and monomeric ingredients until a predetermined monomer is reached.

Related methods for controlling the in vivo functional life of implantable polymeric medical devices include controlling the polymer's inherent morphology.

In one embodiment of the present invention a longer in vivo functional life is provided to a medical device by increasing the percentage of polymer having a crystalline morphology as opposed to an amorphous morphology.

In another embodiment of the present invention crystalline versus amorphous polymer morphology in the medical device is controlled using annealing temperatures and time.

In yet another embodiment of the present invention the present invention crystalline versus amorphous polymer morphology is controlled using monofilament draw ratio.

According to another aspect of the present invention, methods for producing biocompatible, bioresorbable stents having variable in vivo functional lives are provided wherein the ratio of monomer to high molecular weight polymeric sub-units in the polymer material used to form the polymeric stents is adjusted to achieve the desired in vivo functional life.

Additional objects and advantages of the present invention and methods of making same will become readily apparent to those skilled in the art from the following detailed description, wherein only the preferred embodiments are shown and described, simply by way of illustration of the best mode contemplated of carrying out the invention. As will be realized, the invention is capable of modification in various respects, all without departing from the invention. Accordingly, the drawings and description are to be regarded as illustrative in nature, and not as restrictive.

### BRIEF DESCRIPTION OF THE DRAWINGS

A detailed description of the invention is hereafter described by non-limiting examples with specific reference being made to the drawings in which:

FIG. 1 graphically depicts compression resistance of PLLA stents as a function of polymer viscosity over time in accordance with the teachings of the present invention;

FIG. 2 graphically depicts compression resistance of PLLA stents as a function of polymer viscosity over time in accordance with the teachings of the present invention;

FIG. 3 graphically depicts compression resistance of PLLA stents as a function of polymer viscosity over time in accordance with the teachings of the present invention;

FIG. 4 schematically depicts the manufacturing process for woven polymeric stents made in accordance with the teachings of the present invention;

FIG. 5 schematically depicts the manufacturing process for injected molded or extruded tubular polymeric stents made in accordance with the teachings of the present invention;

FIG. 6A is a side view of the bioresorbable stent made in accordance with the teachings of the present invention.

FIG. 6B is an end view of the bioresorbable stent made in accordance with the teachings of the present invention.

FIG. 6C is a perspective view of the bioresorbable stent made in accordance with the teachings of the present invention.

FIG. 7 is a side view of an alternate embodiment made in accordance with the teachings of the present invention.

FIG. 8 is an enlarged view of a partial segment of the bioresorbable stent made in accordance with the teachings of the present invention.

FIG. 9 graphically depicts the bilateral self-expansion force of an alternate embodiment made in accordance with the teachings of the present invention versus UroLume® stents.

FIG. 10 graphically depicts the bilateral compression resistance of one embodiment made in accordance with the teachings of the present invention versus UroLume® stents.

FIG. 11 graphically depicts the radial self-expansion force by a Cuff Test of one embodiment made in accordance with the teachings of the present invention versus UroLume® stents.

FIG. 12 graphically depicts the radial compression resistance by a Cuff Test of one embodiment made in accordance with the teachings of the present invention versus UroLume® stents.

FIG. 13 graphically depicts the bilateral self-expansion force of one embodiment made in accordance with the teachings of the present invention as a function of in vitro aging time.

FIG. 14 graphically depicts the bilateral compression resistance of one embodiment made in accordance with the teachings of the present invention as a function of in vitro aging time.

FIG. 15 graphically depicts the radial compression resistance of an alternate embodiment made in accordance with the teachings of the present invention versus a UroLume® stent.

FIG. 16 graphically depicts the radial self-expansion force of an alternate embodiment made in accordance with the teachings of the present invention versus a UroLume® stent.

FIG. 17 graphically depicts the bilateral compression force versus calculated lumen area of bioresorbable stents made in accordance with the teachings of the present invention.

FIG. 18 graphically depicts the bilateral compression resistance as a function of time in vitro of various embodiments of bioresorbable fenestrated tube stents made in accordance with the teachings of the present invention.

FIG. 19 graphically depicts the bilateral self-expansion force as a function of time in vitro of various embodiments of bioresorbable tube stents made in accordance with the teachings of the present invention.

FIG. 20 schematically depicts the extrusion process used to make the monofilaments in accordance with the teachings of the present invention.

### DEFINITION OF TERMS

Prior to describing the present invention in detail, the following terms will be defined as used herein. The definitions provided immediately below will serve as the intended meaning in this specification and claims even when the following definitions may contradict their ordinary meanings.

Biocompatible: A compound, composition of matter or device made therefrom that does not provoke more than a mild foreign body reaction in the host.

Resorbable/Bioresorbable/Biodegradable: A material that is broken-down in the body of the recipient into normal or non-toxic metabolic by-products. The resulting metabolic by-products are absorbed by the tissues and excreted from the body. A portion of the material may not be absorbed but rather be excreted in whole or in part by a physical action of the body such as peristalsis or urination without physical damage or toxic consequences to the recipient. Portions may also be resorbable. The terms bioresorbable, resorbable and biodegradable may be used interchangeably when describing certain embodiments of the present invention. Unless specifically contradicted by the text, no distinction is to be made between these terms when used in conjunction with urethral stents.

Implantable/Implant: Mechanically or surgically placed into the body of the recipient.

Polymeric sub-units: A monomer, dimer, or oligomer of the basic polymer chain.

Polymeric Ingredients: Polymeric sub-units.

Polymeric composition: A polymeric material composed of at least one polymeric ingredient of at least one type of polymer.

Short to Medium Term Use: The stents of the present invention are intended for in vivo use ranging from 1-3 months for “short-term” applications and 3-6 months for “medium-term” applications.

In vivo functional life: The point at which a polymeric stent has less than 50% of its initial compression resistance as measured in Newtons.

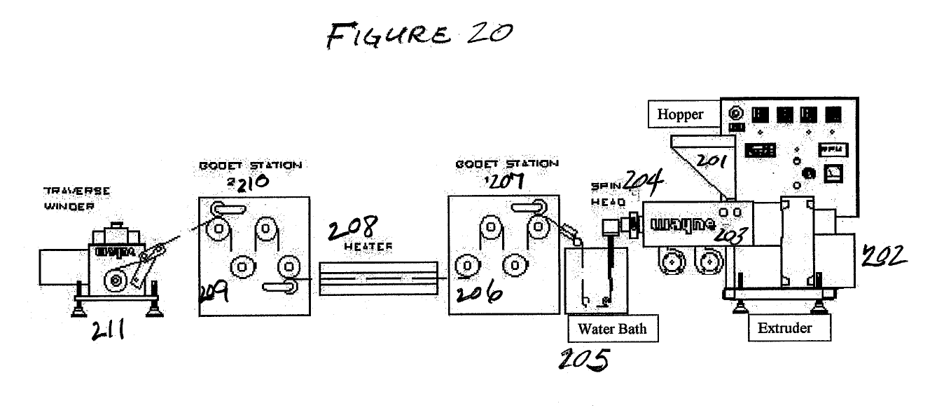

Draw Ratio: This is the ratio of the roller speed at the last godet station to that of roller speed at the first godet station as depicted in FIG. 20.

High molecular weight polymer: A polymer having an inherent viscosity greater than 4.5 dl/g.

Low molecular weight polymer: A polymer having an inherent viscosity less than 4.5 dl/g.

### DETAILED DESCRIPTION

The present invention relates to polymeric medical devices that are implanted into the body of a patient in need thereof. The medical devices of the present invention are designed to be biocompatible and bioresorbable. Biocompatibility is required to enable the medical device to remain in the patient for a sufficient time to provide its intended benefit without provoking an adverse host response. Biocompatibility is achieved by selected materials that are relatively inert, or that are recognized by the host as “self.” For example, many metals are chemically and biologically inert. Examples include stainless steel, titanium nickel alloys and mixtures thereof. Inert materials may also include polymers, or “plastics” that are made from a wide variety of monomeric sub-units.

Many successful implantable medical devices have been made from both metal alloys and polymeric materials. The choice of material is largely predicated on the intended application. Long term medical devices intended to provide the recipient with protection from impact or structural support are generally made from metal alloys. These include, for example, skull plates, artificial-joints, supports for damaged bones and bone screws. However, for many applications metal alloys may be too bulky, rigid or subject to chemical attack and encrustation. Furthermore, medical implants made from metal alloys must either be permanently implanted, or surgically removed. There are many applications where temporary applications are preferred. In these cases, medical devices made from bioresorbable materials that will not require post implantation surgical removal may be preferred.

Bioresorbable medical polymers were first used in the 1970s when resorbable sutures made from Dexon® where introduced. Dexon® is poly-glycolic acid polymer (a poly-alpha-hydroxy acid) composed of glycolic acid sub-units. Poly-glycolic acid (PGA) polymers are degraded in the body by hydrolysis into oligomers that in turn are broken down into glycolic acid monomers. These glycolic acid monomers are ultimately broken-down into pyruvic acid and finally metabolized into carbon dioxide and water. Since the successful introduction of Dextron@ many other biocompatible, bioresorbable polymers have been used to make medical devices.

There are numerous factors that must be considered when selecting a polymer material for use as a medical implant. Structural strength of the implant, duration of implantation, compatibility with host tissues and ease of manufacturing are just a few of the considerations. A wide variety of surgical procedures and applications are contemplated herein. The present invention is believed particularly suitable for use in conjunction with surgical procedures for treating the prostate or the lower urinary tract. For example, a patient undergoing brachytherapy may have a short term stent implanted to resist blockage of the urinary tract due to swelling of the prostate. As another example, a procedure for treating the prostate (e.g. a Trans-Urethral Resection of the Prostate [TURP], microwave therapy, RF treatment, or the like) may also include the implantation of a short term stent before, during or after the procedure. In another embodiment, the stent may be used in conjunction with a treatment for a urethral stricture to help resist any tendency for the tissue to grow together or occlude the urinary tract.

The urethral stents of the present invention are intended for short to medium term applications. Therefore, in one embodiment of the present invention, the stents are made from a polymeric composition designed to be resorbed within in a specified time period. However, in order to provide the recipient its intended benefit, the stent must retain sufficient structural integrity to maintain a minimum compression resistance over its intended in vivo life span. Therefore, resorption must occur gradually. Consequently, the present inventors have developed stents having specific polymer compositions and structural features that fulfill the combined objectives of short to medium term structural strength with bioresorbability.

Physical properties of polymers are influenced by the size of the molecules and by the nature of the primary and secondary bond forces. The type and size of monomers, polymer sub-units, overall polymer viscosity and polymer morphology influence these properties. The present inventors have determined that a polymer's in vivo bioresorption rate and structural strength are a function of these physical properties.

Monomer content can significantly affect in vivo functional life. Specifically, increasing the monomer content in polymeric medical devices made from polymers having high initial molecular weights significantly shortens in vivo functional life. Moreover, polymer morphology also contributes to bioresorption rates. While not as significant as the monomer percentage in the final polymeric composition, the present inventors have demonstrated that increasing the device's amorphous domains relative to its crystalline domains can decrease the polymer's in vivo functional life.

The synthetic polymers of the present invention are produced by a process governed by random events. As a result, the chain lengths of individual polymer sub-units vary. Consequently, a particular polymeric material cannot be characterized by a single molecular weight. Instead, a statistical average of all of the polymeric sub-units is used to denote molecular weight. The molecular weight of polymers can be expressed in different ways including number average, weight average and viscosity average. Number average is the sum of all molecular weights of the individual molecules present divided by their total number. In weight averages each polymeric sub-unit contributes according to the ratio of its particular molecular weight to the total.

For example, imagine a sample having five polymeric sub-units of molecular weight 2, 4, 6, 8 and 10 respectively. To calculate the number average molecular weight, all weights of the individual polymeric sub-units are added. The sum is then divided by the total number of molecules in the sample, in this case 5. Mₙ=2/5+4/5+6/5+8/5+10/5=6. To calculate the weight average molecular weight of the above sample, the squares of each individual weight are divided by the total sum of molecular weights, in this case 30. M_{w}=2²/30+4²/30+6²/30+8²/30+10²/30=7.33. Generally speaking, weight average is more sensitive to the higher molecular weight species and number average is more sensitive to the lower molecular weight species; however, the Mₙ value will usually be within 20% of M_{w}.

As a practical matter, neither of these methods is easily applicable over a wide range of polymers and neither is easily adapted to the manufacturing environment. Furthermore, viscosity average is best suited for linear polymers such as those used in the foregoing examples. Therefore, for these reasons, the viscosity average method will be used throughout this specification to determine and denote the molecular weights.

The present inventors have determined that a polymer's monomer content (measured as a percentage of total polymeric subunits in a polymeric medical device) is directly related to polymer stability under hydrolytic conditions. Hydrolytic stability in turn affects bioresorption rates and hence a medical device's in vivo functional life.

Moreover, the present inventors have also determined that hydrolytic stability is also affected by the polymer's morphology. The present inventors have determined that polymer morphology is affected by physical factors such as initial draw ratio, annealing temperature, annealing time and the extent of contraction allowed during annealing.

The following non-limiting examples describe representative methods used in accordance with teachings of the present invention. Example 1 details methods used to determine polymer inherent viscosity. Example 2 provides methods for determining polymer monomer content using nuclear magnetic resonance (NMR) testing. Example 3 teaches a polymer extrusion process. Example 4 details the method used to test bilateral compression resistance of stents made in accordance with the teaching of the present invention. Finally, Example 5 describes the methods used to simulate the in vivo hydrolytic environment. Stents incubated under the conditions and for the times described in Example 5 were used to assess polymer performance as a function of time under physiological conditions.

### EXAMPLE 1

### Determination of Inherent Viscosity

Linear polymer solution viscosity relates to average molecular weight and can be used to designate polymer size. Capillary efflux time (t) of a polymer dissolved in an appropriate solvent is measured at constant temperature and compared with the efflux time for pure solvent (t₀) at the same temperature. These values are then used to calculate polymer inherent viscosity. While this example uses poly-L-lactic acid (PLLA) polymer, this is not intended as a limitation. The following example can be used to determine the inherent viscosity for many polymers, specifically linear polymers.

1. Supplies, apparatus and reagents

a) scissors

b) forceps

c) analytical balance (calibrated to four decimal places in grams)

d) 50 ml volumetric flasks and glass stoppers, TC=20° C.

e) black Sharpie marking pen

f) chloroform, Fisher Spectranalyzed® in a Safemore® bottle or similar product

g) Shaker

h) thermometer, 19 to 35° C. with 0.02 degree increments

i) DI water

j) glass beaker, 2000 ml

k) paper towels, KimWipes®

l) eye dropper

m) 50 ml graduated cylinder

n) aluminum foil

o) styrofoam insulating ring to fit the outside of a 2000 ml beaker

p) Lauda D20KP capable of maintaining ±0.02° C.

q) Cannon-Fenske viscometer, size 50

r) Lauda PVS 1

s) computer and Lauda Software, LDVM 4014 Rev.2.44

t) fume hood

2. Preparation of Inherent Viscosity PLLA Specimens

a) Prepare three samples per lot as follows:

b) Tare a clean, dry, labeled (sample ID on each flask with Sharpie pen) 50 ml glass-stoppered volumetric flask on the balance.

c) Cut small portions of PLLA material and slowly add it to the volumetric flask until the weight of the material is equal to 0.0500±0.0050 gm. Record the weight to the nearest 0.0001 gram on the C. S. Lab Test Request Form. Repeat two times.

d) Add approximately 35 ml of chloroform to each of the volumetric flasks using a graduated cylinder. Record the Chloroform lot number on the Form.

NOTE: Keep the graduated cylinder and all other containers of chloroform stoppered. If there is no glass stopper, prepare foil caps to place over all open containers of chloroform by pressing a piece of aluminum foil over open container tops to keep out particulate contamination. Do not use rubber stopper.

e) Place the flasks on the shaker and gently agitate overnight at room temperature.

f) Inspect the flasks for particulate contamination or undissolved PLLA. If contaminated with foreign particulates, dump the sample in an appropriate waste container for chloroform waste and repeat the above sample preparation. If the PLLA is undissolved, shake for additional time. If the samples are dissolved and clear of particulates, proceed as follows.

g) Add approximately 14 ml of chloroform to fill each flask almost to the 50 ml mark. Close the flask with a glass stopper. Thoroughly mix by inverting the flask a minimum of ten times.

h) Prepare a water bath at 20±0.02° C. by half filling a 2000 ml beaker with very cold tap water. Use the thermometer and hot and cold tap water to adjust and maintain the water at 20° C. A styrofoam insulation ring may be used to help maintain water temperature at the 20° C. target.

i) Insert flasks in the 20° C. bath and allow a minimum of 20 minutes for the solutions to come to equilibrium.

NOTE: Do not allow the bath water level to cover the top of the volumetric flasks. Water around the stopper will contaminate the PLLA sample solution.

j) Remove the flask from the water bath and dry the flask with a lint free wipe. Dilute the solution to volume by filling the flask to the mark with chloroform using an eyedropper. Mix. Do not overfill.

k) Inspect the solution visually or with a magnifying glass to ensure the absence of undissolved PLLA and foreign particle impurities.

3. Inherent Viscosity Measurement

a) After thoroughly rinsing and drying (aspirate) the viscometer, measure 10 ml of chloroform in a volumetric pipet. Dispense into the clean viscometer and close the lid. Click on Viscometer (stand) icon of choice at the screen and fill in the sample ID, lot number, operator name, etc. Choose kinematic viscosity and click on start to run the standard chloroform sample, automatically.

Note: The viscometer parameters should be preset with the capillary number (position 1 or 2). Choose the capillary list to alter the selection. Use the capillary constant, K=mm²/S² from the manufacturer's viscometer specification sheet and the manufacturer's device number. The maximum standard deviation is set at 0.20 seconds but is actually 0.20 seconds maximum. The start delay is set at 5 minutes. Two pre-measurements and three recorded measurements are also standard practice.

b) Three of the last three or four measurements of efflux time must all agree within 0.20 seconds. For chloroform standards the efflux time must also be very close to the expected efflux time for that particular viscometer from previous testing. If not, rinse and repeat test or dismantle and clean with warm chromic acid cleaning solution by completely filling the viscometer and allowing it to warm in a beaker of hot water for greater than one hour.

Warning: Do not add water to the cleaning solution. Do not get cleaning solution on you skin or clothes. Wear full protective gear while handling cleaning solution. It is extremely caustic.

c) After more than 1 hour of cleaning time, pour the cleaning solution back into the original bottle. Rinse the viscometer ten times with DI water and drain thoroughly.

d) After more than 1 hour of cleaning time, pour the cleaning solution back into the original bottle. Rinse the viscometer ten times with DI water. Take particular care to ensure that a significant volume of each wash passes through the capillary. Drain thoroughly.

e) Rinse with 10 ml Dehydrated Alcohol at least three times to remove water; then rinse more than three times with chloroform and dry thoroughly. Reconnect viscometer to apparatus. Run a rinse cycle and check standard chloroform again.

f) If you have good results consisting of an average chloroform viscosity efflux time within 0.3 seconds of the previous normal averages, test the sample solution next.

g) Make sure the viscometer is completely dry by aspirating. Add 10 ml of dissolved sample to the viscometer, then close the lid. Click on the viscometer icon. Fill in the parameters relevant to the sample, and choose relative viscosity. Click on the purple book icon to choose the last date/time chloroform standard for the viscometer containing the new sample to be tested. Fill in the weight of the monofilament and the volume (total volume of the flask is always 50 ml). OK. Press start.

4. Results

a) Three results must all agree within 0.20 sec. Record results on data sheet and hand calculate as follows:

$$Inherent\_Viscosity \_ \text{(} dl \text{/} g \text{)} = \frac{[ ln ( \frac{EffluxTime\_Solution}{EffluxTime\_Solvent} ) ]}{2 X ( PLLA\_Sample \_Weight \_in \_Grams )}$$

b) Record and repeat 2 more times (n=3), then report the average IV (g/dl).

### EXAMPLE 2

### Nuclear magnetic Resonance Testing of PLL

A polymer is dissolved in an appropriate solvent and examined by NMR to determine its structure. Resonance areas are measured to determine the percent composition of the polymer, the residual monomer and any significant impurities present. Polylactide can be analyzed in deuterochloroform (CDCl₃).

1. Supplies and Reagents

a) 300 MHz NMR spectrometer, Varian XL-300 or equivalent

b) 5 mm OD NMR tubes, 7″ length

c) Ultrasonic bath (Fischer Scientific)

d) Nitrogen bag with closure (I²R Inc.)

e) CDCl₃ (deuterochloroform)³99.6% D (Cambridge Isotopes or equivalent)

f) TMS (tetramethyl silane), as external or internal reference standard.

2. Sample Preparation

a) Weigh approximately 50 mg polymer and transfer it to an NMR tube. Avoid exposing the sample to ambient air and moisture as much as possible.

b) Using a syringe or appropriate pipet, transfer 600 μl CDCl₃ into the tube. Cap the tube and remove from N₂ bag.

c) Place the tube in ultrasonic bath until polymer is completely dissolved.

3. Instrumental Parameters

a) Spectra may be run at any temperature between 20° C. and 45° C., typically at 35° C. Resonance positions will shift slightly with temperature changes.

i. Spectra are run under quantitative conditions:

To observe pulse widths >/=45° C., a recovery delay time of >/=8 seconds is required.

RESONANCE ASSIGNMENTS: PLA IN CDC1₃

| | | | NO. |
|---|---|---|---|
| REGION | ASSIGNMENT | INTEGRAL LIMITS | OF PORTIONS |
| A | Lactide Monomer | 4.99-5.07 | 2 |
| B | PLA | 5.07-5.29 | 2 |

4. Other Resonances

a) Other resonances which are sometimes observed include lactic acid (1.47 and 4.35 ppm) and lactyl lactate (1.50 and 4.35, 5.20 ppm). Aliphatic impurities show methylene resonances at 1.28 ppm and CH₃ groups at approximately 0.9 ppm.

b) If the sample and/or the solvent is not dry, a large water resonance is observed which will interfere with the analysis. In. CDCl3, water resonates at approximately 1.5 ppm. The frequency and width of the water resonance shifts as a function of temperature, water concentration and. acidity of the solution. Care should be taken to exclude any water contamination of sample, solvent, and NMR tubes.

5. Analysis

a) The integrated intensity of the area attributed to the methine region of lactide monomer between 4.99-5.07 ppm (A) is determined and compared to that of the sum of the intensities of the polymer and the monomer. The monomer weight percentage is calculated from the following equation:

$$Monomer ( Wt . \% ) = \frac{A}{A + B} \times 100$$

### EXAMPLE 3

### Extrusion Process (FIG. 20)

Polymer granules are loaded in the hopper 201 of the extruder 202. The extruder screw 203 in the heated barrel melts the polymer and delivers it to metering pump (not shown) under pressure. The metering pump pushes the melt through a spin head 204. A spin head consists of a ‘screen pack’ to filter the melt and a spinneret die. Molten monofilament strands are quenched in a water bath 205.

The quenched strands pass over the rollers 206 of the first godet station 207. The speed of the rollers 206 of godet station 207 is adjusted to match the flow rate through the spin head 204.

The strands then pass through a drawing oven 208 and subsequently over the rollers 209 of the second godet station 210. The speed of the rollers 209 at godet station 210 is faster than roller 206 at the first godet station 207 to apply initial draw to the monofilament strands.

The strands then pass through the next set of drawing oven and godet station (not shown). The rollers at this godet station rotate at even higher speed to apply additional draw to the strands. The strands are next collected over spools on traverse winder 211.

### EXAMPLE 4

### Compression/Relaxation Testing

Bi-lateral compression/relaxation (BLCR) testing used to determine the compression resistance and self expansion force of polymeric stents made in accordance with the teachings of the present invention.

1. Supplies, Apparatus and Reagents

a) Instron, Model 5565 with Merlin Test Profiler software

b) Instron load cell, 200 lb.

c) Bi-lateral Compression/Relaxation test fixture

d) Caliper (mm., calibrated to two decimal places)

2. Use the Instron, Model 5565, with Merlin Test Profiler for Stent Bi-lateral Compression/Relaxation Testing

a) Install the 200 lb. capacity load cell in the Instron, Model 5565.

NOTE: Allow the Instron load cell to warm up ≧90 minutes before calibrating the load cell or beginning testing.

b) Install the bi-lateral (BL) test fixture, PN 35202662, with associated couplings, pins and springs.

c) Turn on the Instron and the computer and access Instron Merlin software. Load method BLCRstnt.

d) Calibrate the load cell after a minimum of 20 minutes of warm up time by clicking on the load cell icon at the top right side of the screen and following the printed instructions to complete the calibration procedure.

e) Verify Profiler parameters by clicking on the stop light icon at the right side of the screen. Check for BL175 mm profiler method at the left side of the screen or hit “Browse” to find and select that Profiler method. Click on “Profiler” and verify the following, then exit Profiler: Tensile Extension, Relative ramp, 1 Ramp, #1, Delta at 10.50 mm, and Rate at −5.0 mm/min. Click on the right pointing arrow at the top of the screen to verify subsequent blocks or ramp parameters as follows: Tensile Extension, Hold, 2 Hold, #2, Duration: 1 minute. Tensile extension, Relative ramp, 3 Ramp, #3, Delta at 10.50 mm, and Rate at 2.0 mm/min. Tensile extension, Hold, 4 Hold, #4, Duration: 1 second. Tensile Extension, Relative ramp, 5 Ramp, #5, Delta at 10.50 mm, and Rate at −5.0 mm/min. Tensile Extension, Hold, 6 Hold, #6, Duration: 1 minute; Tensile extension, Relative ramp, 7 Ramp, #7, Delta at 10.50 mm, and Rate at 2.0 mm/min.

NOTE: After each block change, the save icon should be pressed or the new parameters will revert to the original settings each time you advance to another ramp or block.

f) Set the gauge length by balancing the load at top left side of the screen, then by placing the two 17.5 mm gauge blocks between the surfaces of the BL test fixture on either side of the centered guide pin. Tap the jog down button when close to touching the gauge block and fine tune with “fine position” wheel on Instron console until the load shows a slightly negative reading (the fixture and gauge block are touching). Set the gauge length by pressing the “Reset GL” button on the Instron console when load is ≧−1.000.

g) Press “jog up” on the Instron console to remove gauge blocks and to make room to load the test stent.

h) Record the following stent parameters: sample ID and length in mm.

i) Verify or calculate and record the test travel distance (TTD)=17.5 mm-7.00 mm=10.50 mm.

j) Install test stent on center guide pin of bottom fixture so that it rests on the base of the bottom fixture—it is necessary to press the braid into place centered on the pin). Press “Balance load” at the top of the screen. Press the “Return” button on the Instron console.

k) Click on the Dog Bone icon at the right of the screen. Click on “Define”. Click on “Name” box. Enter or verify all of the following items: sample ID's, # weeks in vitro, nominal stent length at 14 mm OD (i.e., 1.5, 2.0, 2.5, or 3.0 cm), TTD in mm, “BLCRstnt.” Close “Name” box. Click on “Specimen,” and enter the current Sample ID, relaxed length and relaxed OD. For subsequent samples just update “Specimen” and always press “NEXT” at the top right of the screen before entering sample ID and measurements. Close sample screen.

l) Press “start” on the Instron console to begin the test. When the test is complete, remove the test stent after raising the crosshead.

m) Place the PLLA stent in a labeled plastic bag with two holes punched all the way through the bag. Store under high vacuum for later determination of inherent viscosity.

n) Repeat j to l until all specimens have been tested.

3. Results

The test objective is to characterize the compression resistance and the self-expansion (S-E) force of braided PLLA stents. The raw data of crosshead displacement versus force must be treated to obtain the platen gap versus force data for the stent. The data characterize the two cycles consisting of the following 3 sequential steps:

a) In the first step the stent was compressed to a controlled outside diameter (platen gap) at a controlled speed (crosshead speed=−5.0 mm/minute). This portion of the test characterized the compression resistance of the stent.

b) In the second step, the stent was held in the compressed state for a given duration (hold-time). This portion of the test characterized the force decay or the loss of recovery force.

c) In the third step the constraint on the stent was released at a controlled rate (crosshead speed=2.0 mm/minute). This portion of the test characterized the S-E force of the stent.

The data for platen gap versus force from each sample are to be treated to determine two parameters used to describe the stent's mechanical properties. The two parameters are S-E force in the first cycle and compression resistance in the second cycle. The self-expansion force and compression resistance measured at 10 mm platen gap are reported as representative measures of the respective stent properties.

### EXAMPLE 5

An in vitro strength retention stability test was performed on samples from each lot of PLLA braided stent made in accordance with the teachings of the present invention.

1. Test Samples

All stents will be 3.0 cm long at 14 mm OD.

a) PLLA stents exposed to 35 kGy of gamma irradiation: Six stents from each of three sample groups: 537-34AJ, 537-35AJ and 537-36AJ.

b) PLLA stents exposed to 50 kGy of gamma irradiation: Three stents from each of three sample groups: 537-34AK, 537-35AK and 537-36AK.

2. Test Equipment and Supplies

a) Instron testing machine model 5565 equipped with a 200 lb. Load cell, exp. #5684-4 and Merlin Test Profiler software,

b) Bilateral compression-relaxation test fixture, and

c) Circulating constant temperature water bath with cover (37±1° C.).

d) Glass bottles with screw caps (Wheaton Redi-Pak 8 oz squares with PE lined caps or comparable product).

e) 10 mM Phosphate buffered saline (PBS=10 mM phosphate buffer, 138 mM NaCl, 2.7 mM KCl, pH 7.3). This may be prepared from premixed powder packets available from SIGMA (catalog no. P-3813). The initial pH of the solution will typically be 7.3. This is slightly below the pH 7.4 specified on the SIGMA label, but is acceptable for this test.

3. Test Procedure

a) In Vitro Aging of PLLA Stent Samples:

Six stents will be aged from each of the three lots exposed to 35 kGy of gamma radiation (lot no's 537-34AJ, 537-35AJ and 537-36AJ). Three stents will be aged from each of the three lots treated with 50 kGy of gamma radiation (lot no's 537-34AK, 537-35AK and 537-36AK).

b) Samples from the six test groups will be placed in glass bottles filled with PBS (2 30 mL per stent). Up to six stents of the same test group may be incubated together in a single jar. All samples will then be incubated at 37° C. in a constant temperature circulating water bath. The samples will not be agitated during incubation.

c) Just prior to beginning incubation, dissolved air will be removed using the following procedure: Place bottles with stents and PBS in the vacuum oven. Remove all bottle caps. Close the vacuum chamber and gradually reduce chamber pressure to approximately 0.090-0.095 MPa. As the pressure declines, watch for growth of air bubbles on the stents. Control the rate of pressure change to achieve removal of the air without violent bubbling. Hold samples at this pressure for 10 minutes; then release the vacuum filling the chamber with nitrogen. Dry the bottle threads with lint-free wipes; seal and transfer the bottles to the 37° bath.

d) The pH of the PBS in each bottle will be checked after 4 days. For accurate pH determination the solution and pH electrode must both be at room temperature. Approximately 10 mL of PBS will be transferred from each bottle to a tube or vial of suitable size for pH testing. Sufficient time will be allowed for equilibration to room temperature, then pH will be measured with the pH meter.

e) If the pH is below 7.0, the solution in the bottle will be replaced with fresh PBS. Fresh PBS will be pre-warmed to 37° C. The initial buffer will be decanted, discarded, and replaced with an equal volume of fresh PBS.

4. BLCR Testing:

Samples in each group were removed from the saline at weekly intervals and tested for residual strength using the bilateral compression-relaxation test procedure as described in Example 2 above.

5. Storage of Samples after BLCR Testing

When the test on each stent is completed, the stent will be placed in a plastic bag labeled with the appropriate laboratory notebook and page number, stent lot number, and date. Ensure the plastic bag has at least two holes punched through both sides. Store the specimens under high vacuum for later determination of inherent viscosity.

6. Data Conversion

The raw data of crosshead displacement versus force will be converted to platen-gap versus force for each stage of the BLCR test.

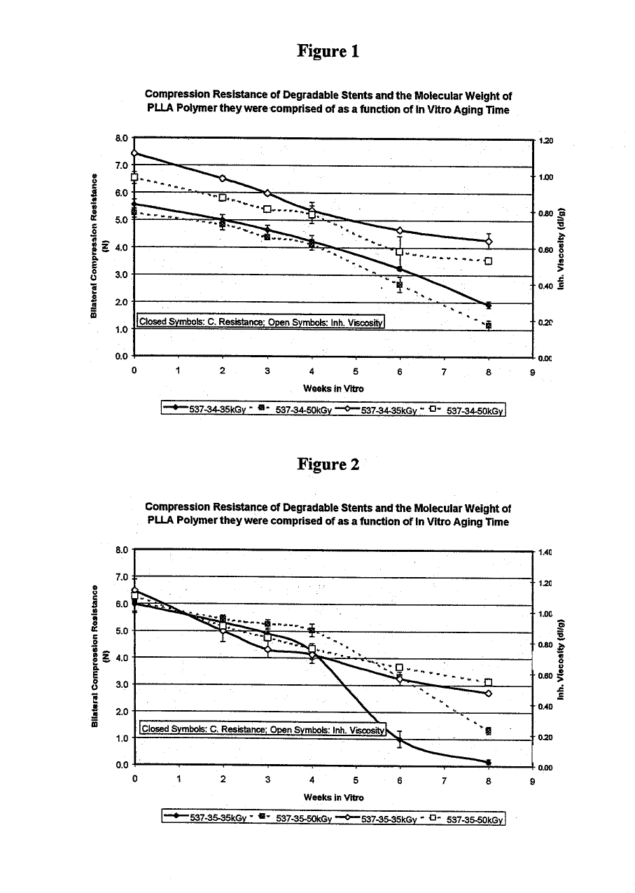

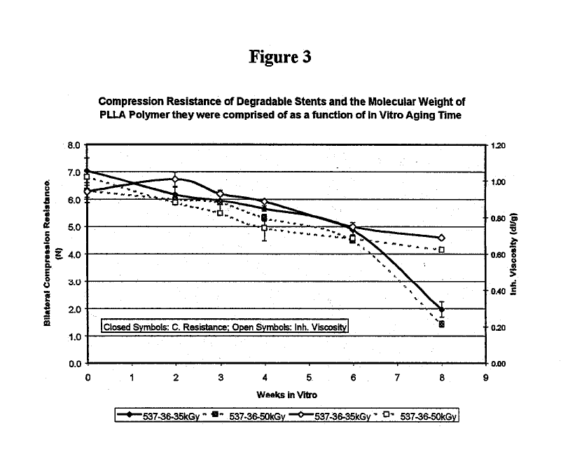

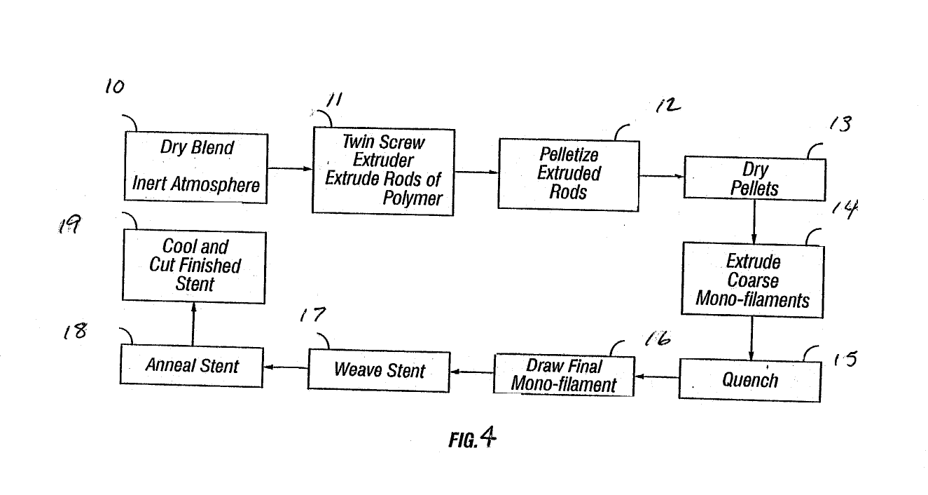

FIGS. 1-3 plot the mean values for the stent samples used in Example 5 and tested in accordance with the teachings of Example 4. FIGS. 1-3 demonstrate a direct correlation between increasing levels of gamma radiation used to treat stent samples and a reduction in initial inherent viscosity and compressive strength. Furthermore, FIGS. 1-3 also demonstrate that as the stents are maintained under simulated in vivo conditions, compressive strength diminishes over time in a direct relationship to reduction in overall inherent viscosity.

The present inventors have determined that there are numerous factors that influence the size distribution of polymeric molecules in a polymeric composition. Specifically, the present inventors have identified several physical factors that can be used to control the monomer content in the polymeric medical devices of the present; invention. For example, and not intended as a limitation, physical processes such as extrusion pressures, and exposure to elevated temperatures can increase the ratio of monomeric sub-units to high molecular weight molecules in polymeric compositions of the present invention. Another method for increasing the monomer content in a polymer is to blend monomer lower sub-units with high molecular weight molecules when formulating the polymer mixture.

As known to those of ordinary skill in the art of polymer chemistry and in accordance with the teachings of the present invention, polymers used to fabricate implantable medical devices can be derived in a number of different ways. In one embodiment of the present invention a polymer is selected having monomer content within a predetermined range. The polymer is then pelletized, milled and extruded into the appropriate configuration. In another embodiment, the polymer is a blend of polymer compositions selected from a number of different molecular weights. The mixture is then blended, pelletized and then extruded.

As used herein, the term “predetermined range” is defined as a value selected based on the teachings of the present invention that will result in the medical device having the functional qualities desired. For example, and not intended as a limitation, a urethral stent having a compression resistance in Newtons (N) of 7.0 with a useful in vivo life span of five weeks is desired (the useful in vivo life span in the present example is defined as the time at which the stent will have a minimum compression resistance in N of <3.5). Based on the teachings of the present invention, it is determined that a polymer stent made using a high molecular weight (high inherent viscosity) polymer and having a monomer content 1.2 weight percent (wt. %) to 2.0 wt. % would be required. This range in monomer wt. % would be the “predetermined range.”

As defined above, a medical device's useful in vivo life span is principally determined by the time it takes to lose 50% or more if its initial structural strength. In the present examples polymeric urethral stents will be the medical device and “structural strength” will be measured by the stent's ability to maintain lumen patency for a specific period (compression resistance as measured by the BLCR test of Example 4). Therefore, in the discussion that follows a stent's structural strength will be its compression resistance measured in Newtons. Therefore, a stent's in vivo functional life is defined as the amount of time an implanted stent will retain at least 50% of its initial compression resistance once exposed to a hydrolytic (in vivo) environment.

The stents of the present invention are intended for short to medium term use. The average in vivo functional life for the bioresorbable stents of the present invention range from approximately 1-3 months for “short-term” applications and 3-6 months for “medium-term” applications. A polymeric stent's structural strength diminishes in vivo as a result of hydrolytic activities. Basically, bioresorbable polymers possess regions within the polymer matrix that are subject to attach by water under physiological conditions. As the polymer matrix undergoes hydrolytic attack, it is broken down into smaller polymeric subunits that are eventually metabolized at the cellular level through the citric acid cycle into water, carbon dioxide and energy. Thus the polymer matrix is weaken by the combined processes of fragmentation and net polymer viscosity reduction. The present inventors have ascertained that there are two fundamental polymer properties that can be modulated during the manufacturing process to control the rate of hydrolytic attack.

The present inventors have discovered that when a high molecular weight polymeric starting material is treated to increase its monomer subunit content the in vivo functional life of the corresponding medical device is shortened. For example, Table 1 depicts the in vitro functional lives of woven urethral stents made from extruded poly-L-lactic acid (PLLA) monofilaments in accordance with the teachings of the present invention. The stents were subjected to in vitro stability testing as detailed in Examples 4 and 5 above. For example, Table 1 demonstrates that stents fabricated using polymers having an initial inherent viscosity of 8.0 dl/g or above lose compression resistance more rapidly as the monofilament monomer content increases (providing the annealing conditions are constant).

Polymer morphology also affects polymeric stent in vivo functional life. Polymeric compositions may be primarily crystalline, amorphous or a combination thereof. Crystalline polymers are generally composed of symmetrical polymer chains that permit the individual polymer molecules to stretch out straight and align themselves with each other. It is well known in the art of polymer chemistry that most polymers do not fully stretch out, but rather are composed of molecules that fold back on themselves forming structures known as lamellae. This is particularly true for high molecular weight polymeric subunits that have a great deal of intramolecular symmetry such as high viscosity PLLA. The lamellae form neatly packed polymer crystals that are tightly packed and resist hydrolytic attack because water does not easily penetrate the hydrophobic regions of the polymer molecule. However, most crystalline polymers may have amorphous regions formed by portions of the polymer chain that do not readily align themselves with the lamellae. The amorphous regions are not susceptible to hydrolytic attack. Therefore, the more amorphous regions in a polymer, the faster it may be degraded in a hydrolytic environment.

It has been determined that the dominant factor affecting in vivo hydrolytic degradation is the percent monomer content and the molecular weight (inherent viscosity) of the pre-processed polymer component. Specifically, the present inventors have ascertained that short and medium term in vivo functional lives are most effectively controlled using a high molecular weight polymer (8.0 or greater dug) as the starting material and increasing monomer content in the final polymer composition. According to the teachings of the present invention, monomer content in the final polymer composition (e.g. a monofilament or stent) can be increased using a number of methodologies.

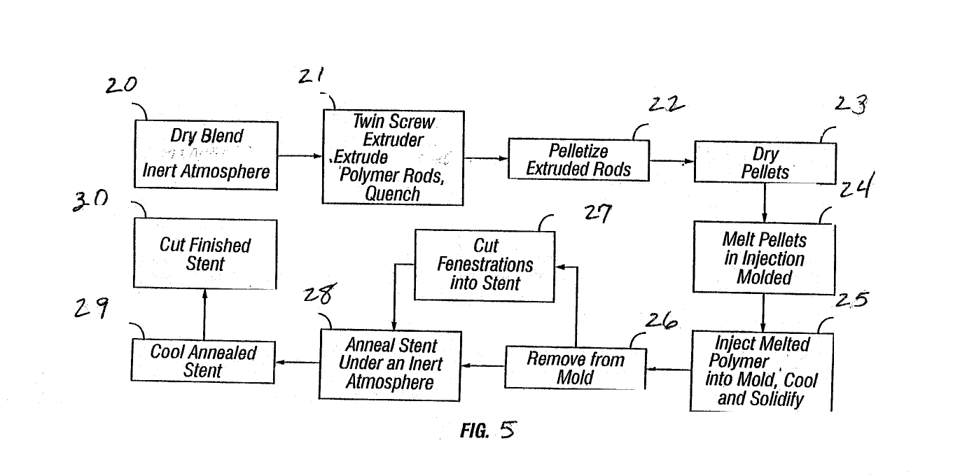

There is essentially phase in the manufacturing of the present medical devices wherein the polymer composition's monomer content may be altered to achieve a predetermined range. This is referred to as the “pre-formation phase.” Referring to FIGS. 4 and 5, “pre-formation” steps include, but may not be limited to, dry blending (10/20), extruding polymer rods (11/21), pelletizing extruded rods (12/22), drying pellets (13/23), extruding coarse monofilaments (14), melting pellets in injection molder (24), Dry quenching (15), injection molding (25), drawing the final monofilament (16), and unmolding (26). The medical devices of the present invention can therefore be fabricated to have a final monomer content within a predetermined range.

Pre-formation steps also include determining the selected polymer's inherent monomer content using methods known to those skilled in the art of polymer chemistry. In one embodiment of the present invention monomer content is determined using NMR techniques. Next, the monomer content of the starting material is compared to the predetermined monomer content for the monofilament or finished stent having the in vivo functional life desired (the predetermined range). If the monomer content is below the predetermined percentage, monomer content is adjusted using one or more pre-formation techniques. In one embodiment of the present invention monomer content is adjusted by adding monomer to the polymer prior to the blending or extrusion processes. In another embodiment polymer extrusion conditions is used to increase monomer content in the polymer composition. For example, extruding a polymer through a small orifice under high pressure will increase monomer content.

In one embodiment of the present invention, a bioresorbable stent is provided having an initial compression resistance of 6 N and a useful in vivo life of eight weeks. Consequently, if the initial polymer selected to make this particular stent has an initial inherent viscosity of 8.0 dl/g, then it can be determined from Table 1 that the monomer content of the pre-annealed, pre-irradiated polymer must be below 1.4%. Preferably the monomer content is between approximately 1.1 and 1.31%. Therefore, the polymer composition used in this non-limiting example may be prepared by blending high molecular weight PLLA preparations to obtain the predetermined monomer range or a high molecular weight PPLA may be extruded at a pressure such that the predetermined amount of PLLA monomer is formed in the polymer composition prior to completing stent fabrication. Alternatively, a combination of methods may be used to achieve the predetermined monomer content.

Additionally, stents made in accordance with the teachings of the present invention may be treated after fabrication in order to achieve desired bioresorbability rates. For example, these post fabrication processes include, but are not limited to, exposing the finished stent to different doses of gamma irradiation from a Cobalt 60 source and/or annealing the stent at different temperatures and for different times.

Regardless of the method selected to adjust monomer content in the final bioresorbable stent, monomer content can be monitored through out the manufacturing process to verify that the predetermined monomer content is achieved. Furthermore, using the teachings of the present invention, and combined with skills known to those in ,the art of polymer chemistry, the exact monomer content can be achieved by using NMR, or other techniques, to monitor the stent's monomer content during manufacturing (in process testing).

The present inventors have discovered that for any given type of bioresorbable polymeric composition used to make the medical devices in accordance with the teachings of the present invention, the ratio of monomer content to high molecular weight polymeric subunits has the greatest effect on bioresorption rates.

In the case of gamma radiation, the amount of energy used, 35 kGy to 50 kGy respectively is greater than that used for sterilizing medical devices. Generally, 25 kGy (10 kilo Gray [kGy] is equivalent to 1 MegaRad [Mrad] of radiation) is recommended for sterilization of most medical devices. However, as previously explained, higher doses of radiation are used in the present invention randomly decrease the molecular weight of the high molecular weight polymeric sub-units. Moreover, the stents described herein have also been subjected to annealing in order to achieve the initial compression resistance desired. As a result, in vivo functional lifes of the polymeric stents used in the following examples result from the synergistic effects of the polymer's base composition, heat and gamma irradiation. As discussed above, other physical characteristics of the polymeric composition such as, but not limited to, polymer crystalline content versus amorphous content (polymer structure) in the final composition also affect bioresorption rates. Physical factors such as gamma irradiation, extrusion temperature and pressure and draw ratio annealing time temperature can affect polymer structure as well as monomeric content. Therefore, a bioresorbable polymeric implant's functional in vivo life results from synergy between pre- and post fabrication processes and is not the result of a single variable. Moreover, as will be discussed further below, the stent's physical configuration will dramatically affect its overall structural integrity and, thus, in vivo life span. Stents woven from monofilaments have different physical qualities than stents made from solid extruded tubes having fenestrations cut therein. Also, the monofilament diameter as well as the number of stands and braiding pattern have a significant impact on stent strength and, thus, in vivo life.

Ultimately, it is the overall combination of physical, mechanical and chemical properties that define polymeric filaments' final physical properties such as tensile strength and tensile modulus. Tensile strength is defined as the force per unit cross-sectional area at the breaking point. It is the amount of force, usually expressed in pounds per square inch (psi), that a substrate can withstand before it breaks, or fractures. The tensile modulus, expressed in psi, is the force required to achieve one unit of strain which is an expression of a substrate's stiffness, or resistance to stretching and relates directly to the stent's performance.

For example, in one embodiment of the woven stent made in accordance with the teachings of the present invention the filament possesses a tensile strength in the range from about 40,000 psi to about 120,000 psi with an optimum tensile strength for the filament 30 of approximately between 60,000 to 120,000 psi. The tensile strength for the fenestrated stent 23 is from about 8,000 psi to about 12,000 psi with an optimum of about 8,700 psi to about 11,600 psi. The tensile modulus of polymer blends in both embodiments ranges between approximately 400,000 psi to about 2,000,000 psi. The optimum range for a stent application in accordance with the present invention is between approximately 700,000 psi to approximately 1,200,000 psi for the woven embodiment and approximately 400,000 psi to 800,000 psi for the fenestrated embodiment.

The methods for making stents 30 (FIG. 6) in accordance with the teachings of the present invention will now be described (FIG. 4). A single PLLA formulation having a predetermined inherent viscosity may be used alone, or it may be blended with one or more PLLA compositions having different inherent viscosities and/or differing amounts of PLLA monomer. The exact number of steps used to make all possible embodiments of the present invention will vary depending upon the whether polymer blends are used, or whether a single polymer having a predetermined inherent viscosity is used and how much and/or if any additional monomer is added. If two or more polymers are used (including two or more samples of the same polymer each having different mean molecular weights and/or additional monomers) the manufacturing process will begin with dry blending under an inert atmosphere (10 in FIG. 4 or 20 in FIG. 5). For stents made from a single homopolymer or co-polymer without the addition of monomer, the process may begin by extruding polymer rods (11 in FIG. 4 or 21 in FIG. 5) or by adding pellets (13 in FIG. 4 of 23 in FIG. 5) directly to either an extruder (14 in FIG. 4) or injection molder (24 in FIG. 5).

FIG. 4 depicts the basic steps for making one embodiment of the present invention. For woven stents, one or more polymer compositions are selected such that the final monofilament will have monomer content within a predetermined range. Next the polymer composition(s) is dry blended 10 under an inert atmosphere, then extruded in rod form 11. The polymer rod is pelletized 12 then dried 13. The dried polymer pellets are then extruded 14 forming a coarse monofilament which is quenched 15. The extruded, quenched, crude monofilament is then drawn into a final monofilament 16 with an average diameter from approximately 0.145 mm to 0.6 mm, preferably between approximately 0.35 mm and 0.45 mm. Approximately 10 to approximately 50 of the final monofilaments 16 are then woven 17 in a plaited fashion with a braid angle 46 (FIG. 6A), from about 100 to 150 degrees on a braid mandrel of about 3 mm to about 30 mm in diameter. The plaited stent 30 (FIG. 6A) is then removed from the braid mandrel and disposed onto an annealing mandrel having an outer diameter of equal to or less than the braid mandrel diameter and annealed 18 at a temperature between about the polymer-glass transition temperature and the melting temperature of the polymer blend for a time period between about five minutes and about 18 hours in air, an inert atmosphere or under vacuum. The stent 30 (FIG. 6A) is then allowed to cool and is then cut 19.

The manufacturing flow chart of stent 50 (FIG. 7) is presented in FIG. 5. A first step 20 may include blending one or more polymers or a single polymer using multiple inherent viscosities. The blending is done in an inert atmosphere or under vacuum. The polymer is extruded in rod form 21, quenched 21, and then pelletized 22. Typically, the polymer pellets are dried 23, then melted in the barrel of an injection molding machine 24 and then injected into a mold under pressure where it is allowed to cool and solidify 25. The stent is then removed from the mold 26. The stent tube may, or may not, be molded with fenestrations in the stent tube.

In one embodiment of the fenestrated stent 50 (FIG. 7) the tube blank is injection molded or extruded, preferably injection molded, without fenestrations. After cooling, fenestrations are cut into the tube using die-cutting, machining or laser cutting, preferably laser cutting 27. The resulting fenestrations, or windows, may assume any shape which does not adversely affect the compression and self-expansion characteristics of the final stent.

The stent is then disposed on an annealing mandrel 28 having an outer diameter of equal to or less than the inner diameter of the stent and annealed at a temperature between about the polymer-glass transition temperature and the melting temperature of the polymer blend for a time period between about five minutes and 18 hours in air, an inert atmosphere or under vacuum 28. The stent 50 (FIG. 7) is allowed to cool 29 and then cut as required 30.

Turning now to specific embodiments of the present invention, FIGS. 6A-6C, depict a bioresorbable, self-expanding stent 30. FIGS. 6A-6C show the bioresorbable stent 30 comprising a cylindrical sleeve having a first end 38 and a second end 40. A plurality of monofilaments 32 which are positioned substantially parallel and helically wound about the longitudinal axis 34 of the stent 30 to form a latticed network 35. The latticed network 36 forms the wall 42 of the bioresorbable stent. As shown in FIGS. 6A-6C, the monofilaments 32 are braided in an alternating under-two-over-two pattern forming the latticed network. The braid-crossing angle 46 is the obtuse angle between any two monofilaments 32 at a point of intersection. In the first embodiment of the present invention, thirty to forty-eight monofilaments may be braided to form the bioresorbable stent 30; preferably forty monofilaments are braided to form the bioresorbable stent. The present invention also contemplates braiding patterns such as, but not limited to, under-one-over-one, under-one-over-two, under-one-over-three, under-two-over-three, under-three-over-three, and the like.

Because forty monofilaments are used on a 48 carrier braiding device; uneven openings result as shown in FIGS. 6A-6C. That is, the openings in the latticed network are not uniform. However, those skilled in the art will appreciate that uniform openings may be provided in a bioresorbable stent by manufacturing the stent on a braiding device with the appropriate number of evenly spaced carriers. For example, a thirty-strand stent may be formed on a 30 carrier braiding device. Uniform openings may also be achieved by pairing strands in a 48-strand stent with the under-two-over-two braid pattern.

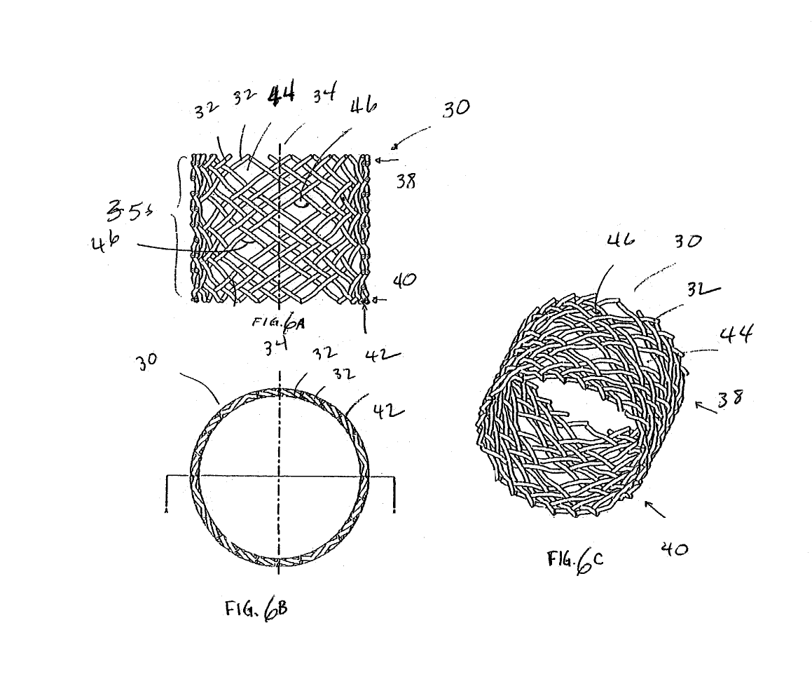

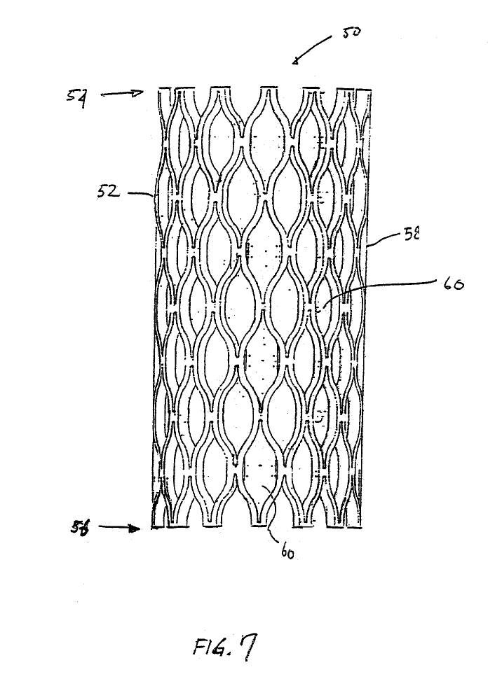

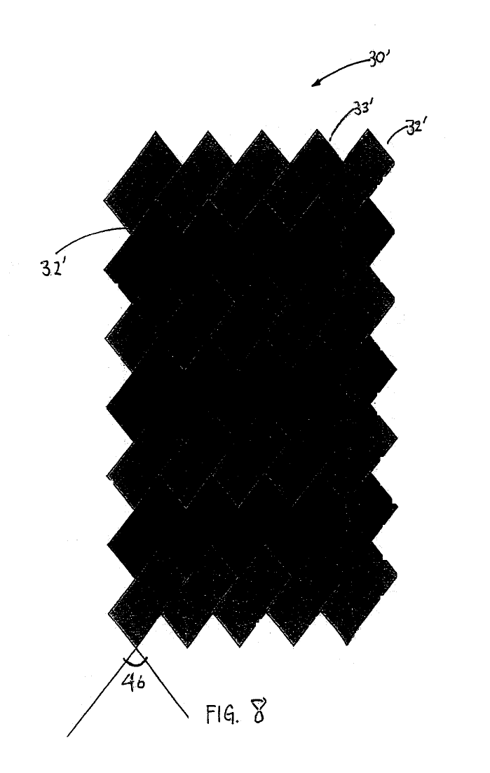

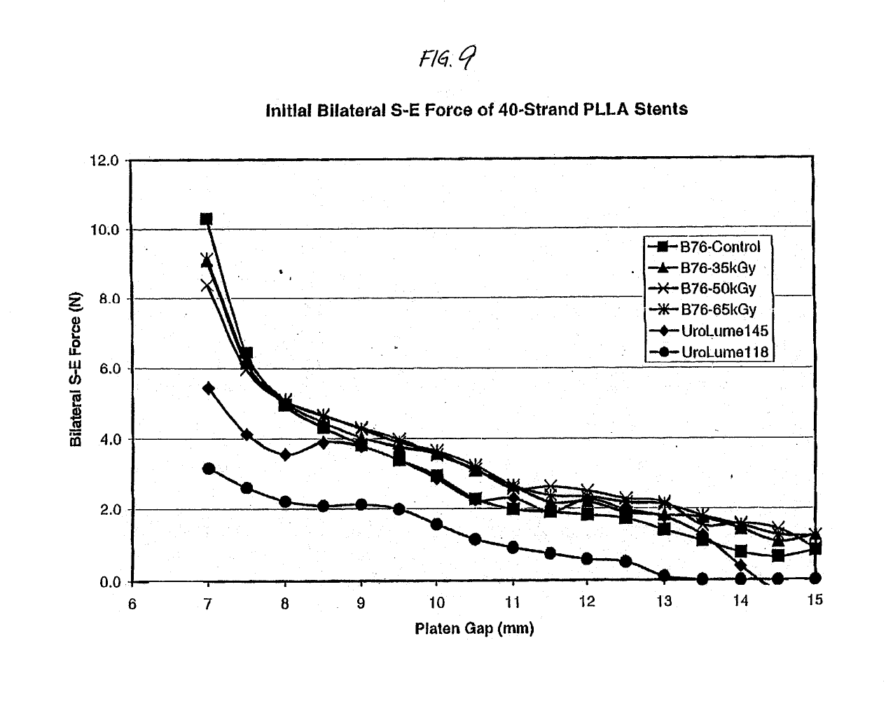

FIG. 8 is an enlarged view showing the under-two-over-two braiding pattern of the bioresorbable stents 30, 30′ of the present invention. Furthermore, FIG. 8 illustrates a bioresorbable stent 30′ having a single strand shift. A single strand shift is defined as adjacent monofilaments 32′, 33′ having a different braiding pattern. For instance, a monofilament 32′ will have an under-two-over-two braiding pattern and the adjacent monofilament 33′ will have an under-two-over-two braiding pattern offset by one monofilament. Stated differently, any adjacent monofilaments will not go “under and over” the same monofilaments.

FIGS. 6A-6C also show openings 44 between the individual monofilaments 32 that comprise the latticed network 35 of the stent 30. Providing spaces throughout the latticed network 35 of the stent 30 allows for sufficient tissue in-growth between the monofilaments of the latticed network thereby fixing the stent in position and minimizing the likelihood of stent migration or dislodgment. Those skilled in the art will appreciate that bioresorbable stents having openings of different sizes are also contemplated in the present invention provided that suitable self-expansion forces and compression resistance are achieved.

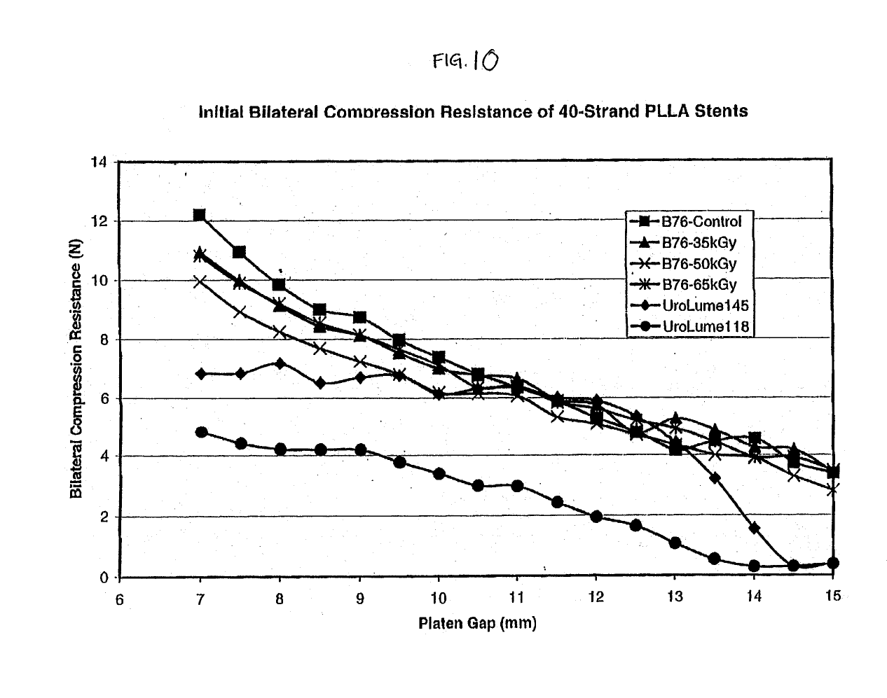

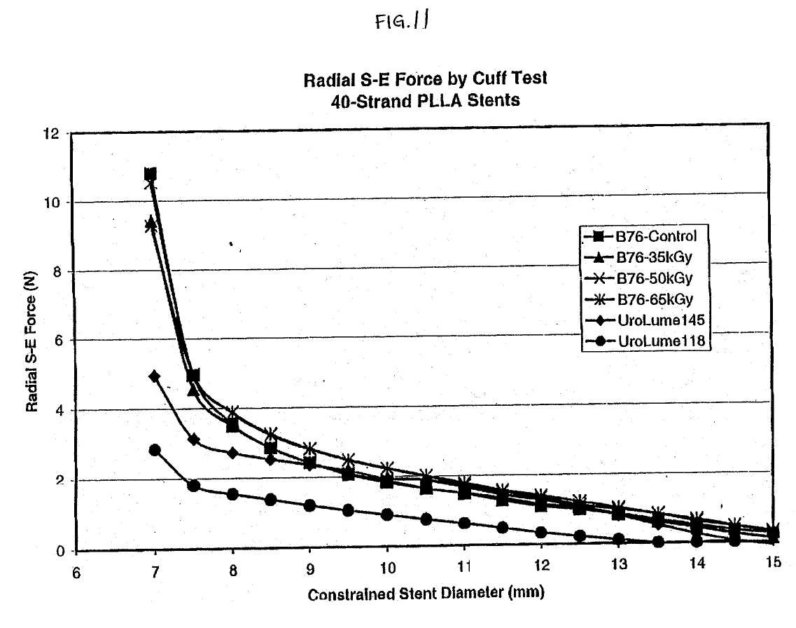

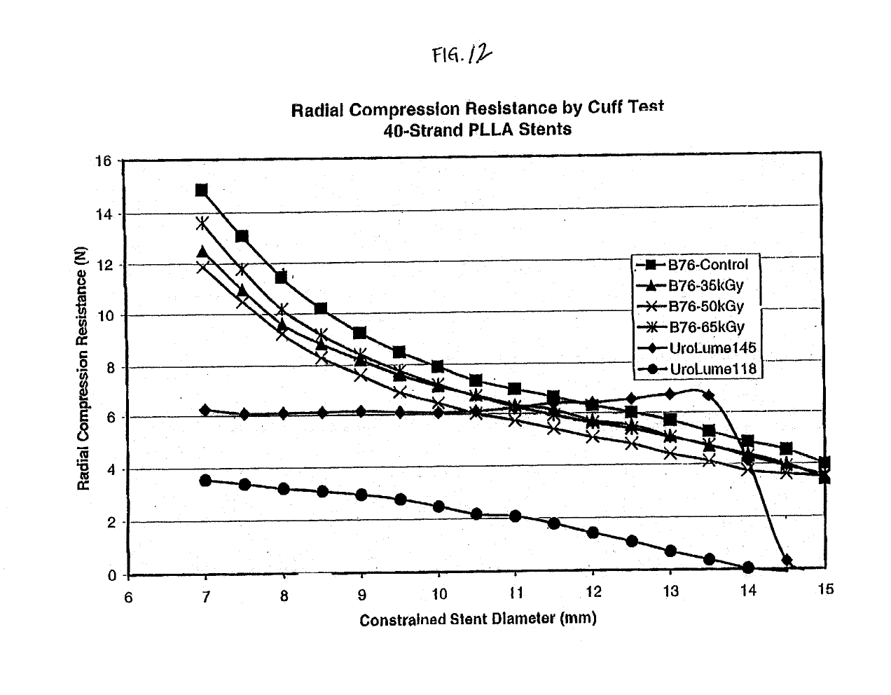

The under-two-over-two braided pattern as well, as other braided patterns of the present invention, is easy to manufacture; yet the braided patterns provide large radial forces as compared to traditional stents. FIGS. 9-10 graphically depict the bilateral self-expansion forces and compression resistance forces of one embodiment of the present invention versus UroLume® stents. UroLume® is the trademark for a metallic stent marketed by American Medical Systems, Inc., the assignee of the current application. In particular, FIGS. 9-10 graphically compare bioresorbable stents having 40 poly-L-lactic acid monofilaments braided in an under-two-over-two pattern and treated at various gamma irradiation doses (35 kGy, 50 kGy, and 65 kGy) versus UroLume® stents having braid-crossing angles of 118° and 145°.

The stent samples were subjected to a bilateral compression-relaxation test using an Instron test machine. The stents were compressed bilaterally between two smooth platens of a Delrin fixture from a resting state to a platen gap of 7 mm. The platen gap range of 7 mm to 15 mm corresponds to the stent diameter in a compressed state (7 mm) and an expanded state (15 mm). The stents were held for a set hold-time of approximately 1 minute, and the stents were allowed to relax. The stents were subjected to two cycles of compression, hold, and relaxation. The force exerted by the stent during the relaxation stage of the first cycle was recorded as the self-expansion force. The force applied to compress the stent in the second cycle was recorded as the compression resistance of the stent.

FIG. 9 illustrates that the bioresorbable stents of the present invention have better bilateral self-expansion forces as compared to the UroLume® stents over a platen gap range of 7 mm to 15 mm. For instance, at a platen gap of 7 mm, a bioresorbable stent exposed to 35 kGy dose of gamma irradiation exerts a bilateral self-expansion force of approximately 9 N while UroLume® stents having braid-crossing angles of 1180 or 1450 exert self-expansion forces of 3N and approximately 5 N, respectively. FIG. 5 shows similar results were obtained when comparing the compression resistance of the bioresorbable stents with the UroLume stents® over a platen gap range of 7 mm to 15 mm. The bioresorbable stents exposed to 35 kGy, 50 kGy, and 65 kGy doses of gamma irradiation demonstrated greater bilateral compression resistance as compared to the UroLume® stents.

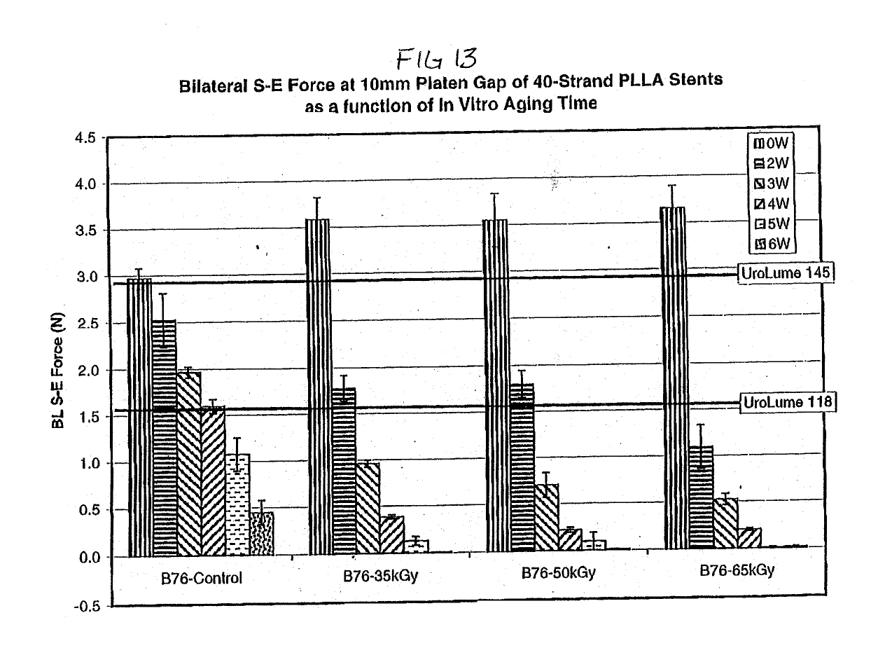

FIGS. 11-12 also show similar results when the stents of the present invention and UroLume® stents were subjected to a Cuff test. The Cuff test was conducted on an Instron test machine using a test fixture and a Mylar® collar. The test fixture consists of a pair of freely rotating rollers separated by a 1-mm gap, and the Mylar® collar is a laminated film of Mylar® and aluminum foil. A 30-mm long stent segment was wrapped in a 25-mm wide collar and the two ends of the collar were passed together through the rollers of the test fixture. A pulling force was applied to the collar ends which radially compressed the stent against the rollers. The stent samples were compressed from their resting diameter to a predetermined diameter (typically 7-mm). The stent samples were compressed and held at the predetermined diameter for approximately one minute, and then they were allowed to relax. The stents were subjected to two cycles of compression, hold and relaxation. The force exerted by the stent during the relaxation stage of the first cycle was recorded as the self-expansion force. The force applied to compress the stent in the second cycle was recorded as the compression resistance of the stent.

The bioresorbable stents of the present invention demonstrated greater radial self-expansion forces over the whole range of constrained stent diameters from 7mm to 15 mm as compared to the UroLume® stents. In particular, the bioresorbable stents displayed approximately 9 N to 11 N of radial self-expansion force at a constrained stent diameter of 7 mm as compared to 3 N and 5 N at 7 mm of radial self-expansion force for the UroLume stents, as shown in FIG. 10. The superior results are also illustrated by the graphical data in FIG. 11.

The graphical data set forth in FIGS. 9-11 illustrate that the bioresorbable stents having an under-two-over-two braided pattern have superior radial self-expanding forces and compression resistance forces as compared to UroLume® metallic stents. Furthermore, the bioresorbable stents of the present invention are also controllably biodegradable which eliminates the need for complicated or invasive stent removal procedures. That is, once an implanted stent has served its intended function, the stent is controllably degraded and naturally eliminated by the human body.

The bioresorbable, self-expanding stents are manufactured by providing a plurality of monofilaments and braiding these monofilaments in an under-two-over two pattern to form a latticed network as shown in FIG. 6 and FIG. 8. As previously stated, it is contemplated that the latticed network of the bioresorbable stents comprises thirty to forty-eight monofilaments. The latticed network is formed by winding the monofilaments about a mandrel. Approximately half of the monofilaments are wound around the mandrel in a clockwise direction while the other half of the monofilaments are wound in a counter-clockwise direction. The angle between the two filaments at the point where they intersect is defined as the braid-crossing angle 46 as shown in FIG. 6. It is contemplated that the monofilaments intersect at a braid-crossing angle between 100° to 150°. In a preferred embodiment, the bioresorbable stents comprise monofilaments having an as-braided braid-crossing angle of 110°. Those skilled in the art will appreciate that other braid-crossing angles may be selected to achieve different self-expansion forces or compression resistance.

The bioresorbable stents then undergo an annealing process. The annealing process includes placing the bioresorbable stents on a mandrel, axially compressing the stents by 30% to 60%, heating the stents to the glass transition temperature of the biocompatible polymer for a predetermined period of time, and allowing the stents to be controllably cooled. The annealing process relieves internal stresses and instabilities of the monofilaments that result from the production of the bioresorbable stents. In a preferred embodiment of the present invention where the latticed structure is formed from poly-L-lactide monofilaments, the bioresorbable stents are heated to approximately 90° C. for a length of time between about one and about eight hours, preferably four hours, in an inert atmosphere. The inert atmosphere may be comprised of a high vacuum or nitrogen gas. Those skilled in the art will appreciate that other inert atmospheres having low moisture content are also contemplated including, but not limited to, argon, or helium. The bioresorbable stents are then controllably cooled to room temperature. Each stent is then cut to desired size for its intended application. Thereafter, the stents are exposed to Co⁶⁰ gamma irradiation to fine tune the in vivo functional life of the bioresorbable stents. Exposure to gamma irradiation causes molecular degradation of the polymers that comprise the bioresorbable stents; however, the gamma irradiation does not affect the overall morphology of the polymers.

During the annealing process, the monofilaments that comprise the bioresorbable stent contract resulting in a different final braid-crossing angle. In contrast to traditional methods where the monofilaments are annealed prior to braiding, the contraction of the monofilaments that comprise the braided stent is important in achieving the compression resistance and self-expansion forces for the stents of the present invention. The final post-annealing braid angle ranges from approximately 125° to 150°, and more particularly a final braid angle ranging from approximately 130° to 145°. Those skilled in the art will appreciate that the final post-annealing braid angle is dependent upon the desired properties and stent length. For instance; a 1.5 cm long stent would require a final post-annealing braid angle ranging from approximately 139° to 145° whereas a lesser braiding angle might be adequate for a longer stent.

The in vivo functional life of the bioresorbable stents is related to the temperature and duration of the annealing process and the dosage of gamma irradiation. Accordingly, the functional lifetime of the stents can be controlled and/or adjusted by manipulating the annealing conditions during the manufacturing process. In one embodiment of the present invention, the annealing conditions of 90° C. for a length of time between about one to about eight hours, preferably four hours, in an inert atmosphere followed by 50 kGy dose of gamma irradiation provides bioresorbable stents having approximately a two week functional life and substantial stent degradation by approximately the fourth week of in vivo implantation. In another embodiment of the present invention, the bioresorbable stents may be annealed at a temperature higher than 110° C. for at least eight hours to achieve an in vivo functional life between three to six months. The bioresorbable stents are typically annealed at 110° C. for approximately eighteen hours to achieve an in vivo functional life between three to six months. Those skilled in the art will appreciate that the annealing parameters may be adjusted for shorter or longer in vivo functional lives.

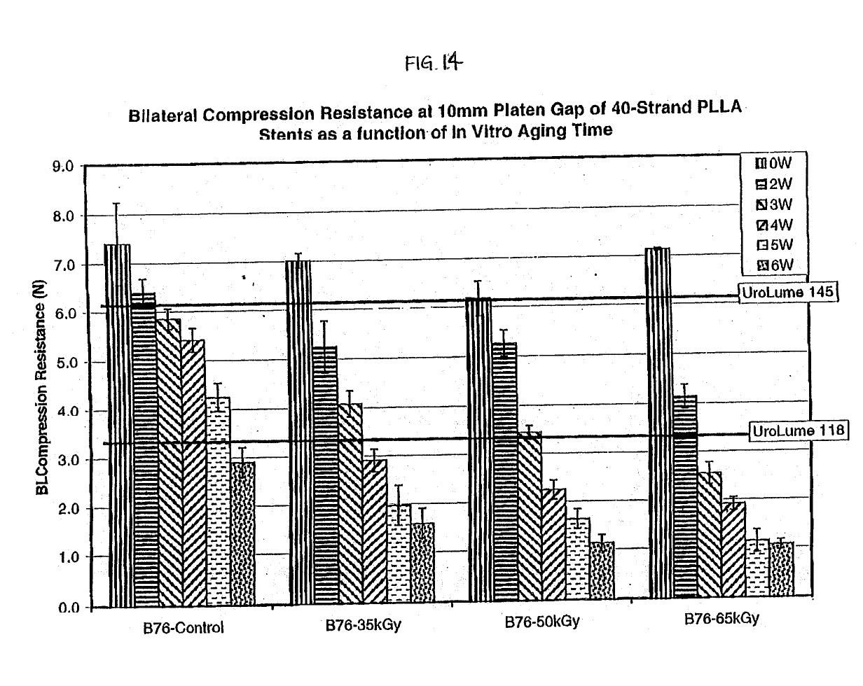

FIGS. 13-14 graphically illustrate the mechanical strengths of the bioresorbable stents of the present invention as a function of in vitro aging time. The in vitro study parameters were designed to mimic in vivo functional life. Accordingly, the stents were aged in a phosphate buffered saline (pH 7.3) at 37° C., and samples were then tested in a bilateral compression/relaxation test at each corresponding aging period. In particular, FIGS. 13-14 show the changes in the self-expansion force and bilateral .compression resistance of the bioresorbable stents over a six week period of time. For instance, as shown in FIGS. 13-14, the stents exposed to 35 kGy and 50 kGy doses of gamma irradiation retained ≧70% of their initial mechanical strength for two weeks, but a substantial degradation in mechanical strength had occurred by the fourth week.

FIG. 7 illustrates a second embodiment of the present invention. The second embodiment of the present invention is similar to the laser cut stent as disclosed in U.S. Pat. No. 5,356,423, the entire contents which are herein incorporated by reference. The bioresorbable stent 50 is comprised of a tubular sheath 52 having a first end 54 and a second end 56. A walled surface 58 having a plurality of fenestrations 60 spaced throughout the walled surface 58 is shown in FIG. 7. The walled surface 58 is contemplated to have a thickness of 0.025″ to 0.030″, preferably 0.030″. The fenestrations 60 are shaped in such a manner to maximize the number of openings for tissue in-growth while maintaining the predetermined self-expansion and compression resistance forces of the bioresorbable stent.

The bioresorbable stents, as shown in FIG. 7, are formed by the following process. Bioresorbable, biocompatible polymers are injection molded or extruded into a tubular sheath. The polymers may be selected from any known bioresorbable polymers including, but not limited to, polyanhydrides, polycaprolactones, polyglycolic acids, poly-L-lactic acids, poly-D-L-lactic acids, polydioxanone, and polyphosphate esters. In a preferred embodiment, polydioxanone is used to form the tubular sheath. Furthermore, it is contemplated that blends or copolymers of the aforementioned biocompatible polymers may be used to form the bioresorbable stents of the present invention. The tubular sheath may be injection molded with or without fenestrations. In a preferred method, the tubular sheath is injection molded without fenestrations. The fenestrations are introduced into the tubular sheaths by cutting processes including, but not limited to, laser cutting and machining.

The bioresorbable stents then undergo an annealing process. The annealing process includes heating the stents to or above the glass transition temperature of the biocompatible polymer for a predetermined period of time, and allowing the stents to cool slowly. The annealing process relieves internal stresses and instabilities that result from the production of the bioresorbable stents of the present invention. Bioresorbable stents made from polydioxanone are heated to a temperature of approximately 75° C. for between about one and six hours, preferably three hours, in an inert atmosphere of high vacuum or nitrogen gas and controllably cooled for approximately twelve hours. Those skilled in the art will appreciate that other inert atmospheres having low moisture content are also contemplated including, but not limited to, argon, or helium.

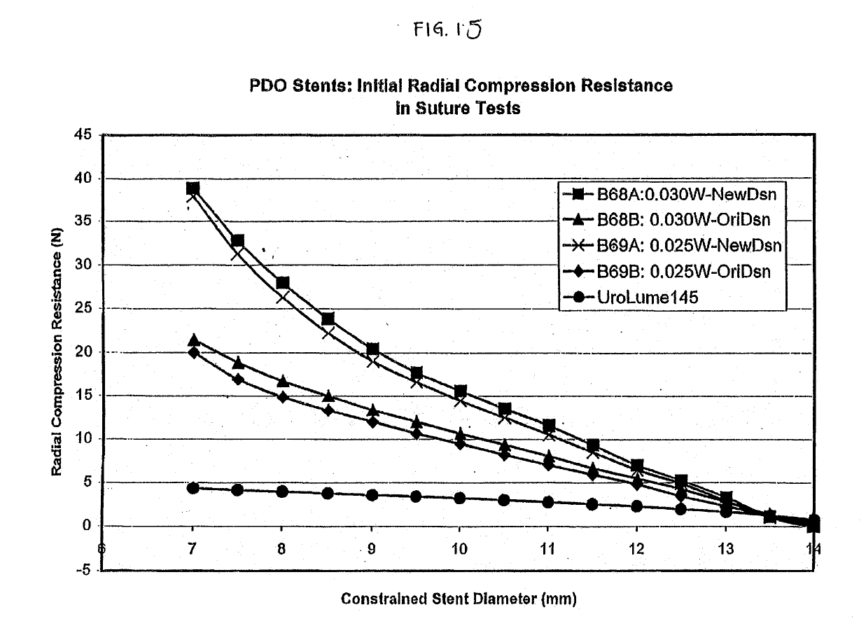

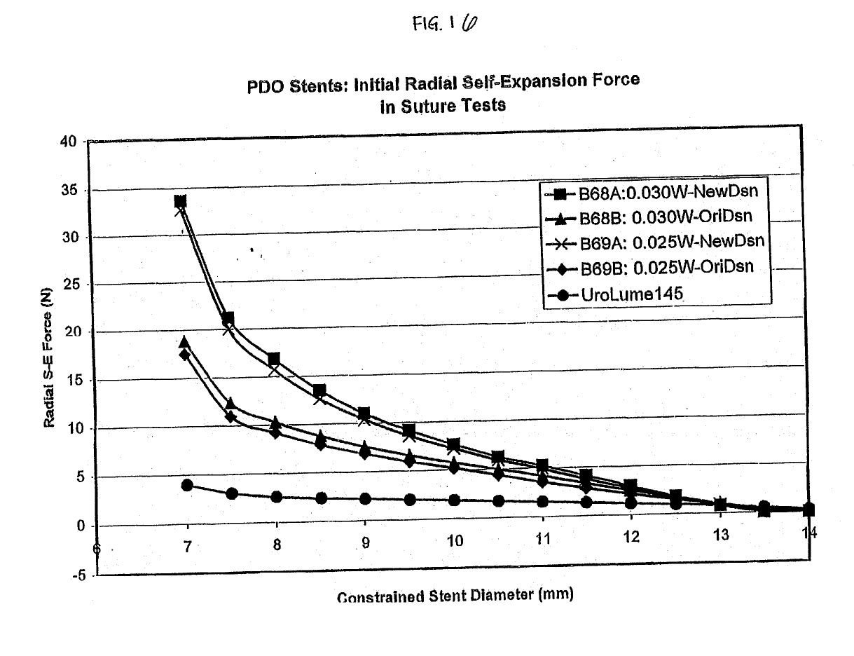

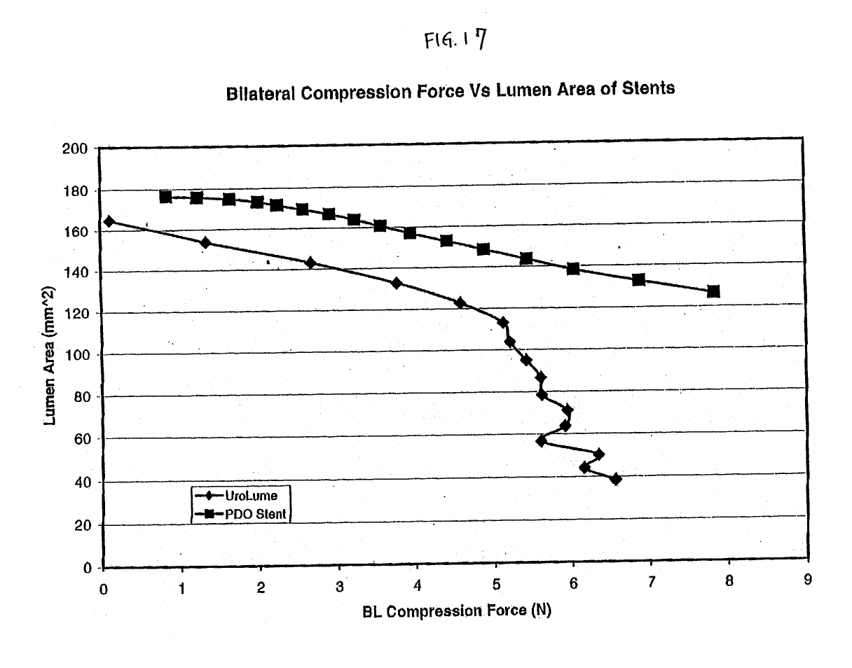

The graphical data set forth in FIGS. 15-16 illustrates the mechanical properties of the bioresorbable stent 50. In particular, FIGS. 15-16 graphically depict the radial compression resistance and self-expansion forces of two embodiments of the bioresorbable stent 50 having different fenestration designs and wall thickness versus a 145° urolume® stent. The stent samples were subjected to a Suture test using an Instron test machine. The Suture test is similar to the Cuff test with the exception that a suture, rather than a Mylar® collar, is used to apply radial compression to the stent and the two ends of the suture are passed through a Delrin guide before passing through the rollers of the test fixture. Like the Cuff test, the stent samples were compressed and held at the predetermined diameter for approximately one minute, and then they were allowed to relax. The stents were subjected to two cycles of compression, hold and relaxation. The force exerted by the stent during the relaxation stage of the first cycle was recorded as the self-expansion force. The force applied to compress the stent in the second cycle was recorded as the compression resistance of the stent.

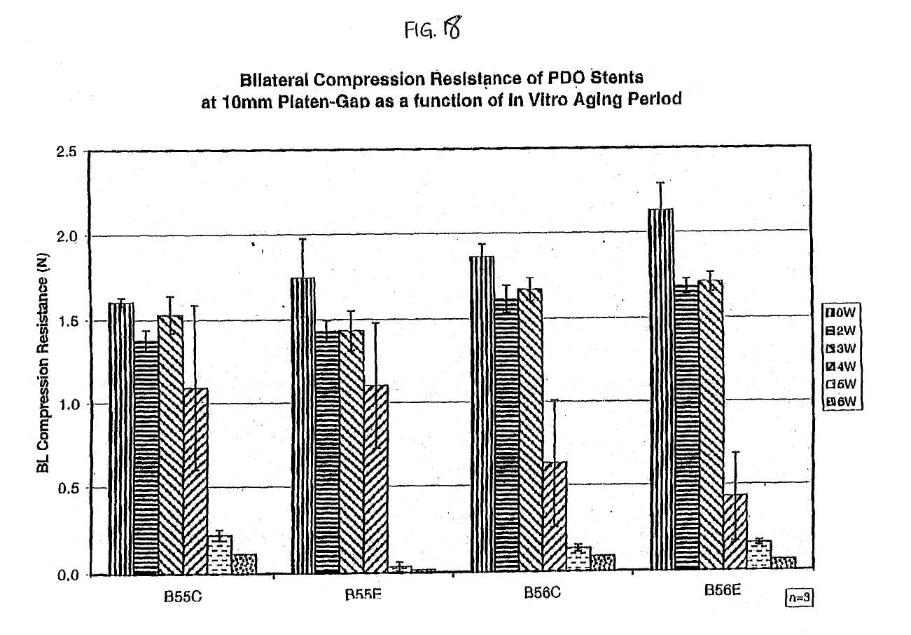

As shown in FIGS. 15-16, the bioresorbable stents of the present invention displayed substantially higher radial mechanical properties as compared to the urolume® stent. FIG. 17 graphically depicts the cross-sectional lumenal area as a function of bilateral compression force for bioresorbable fenestrated tube stents and 145° urolume® stent. FIG. 17 shows that for the same amount of bilateral compression, the reduction in the lumen size of a urolume® metallic stent was significantly greater than that of the bioresorbable stent 50 of the present invention.

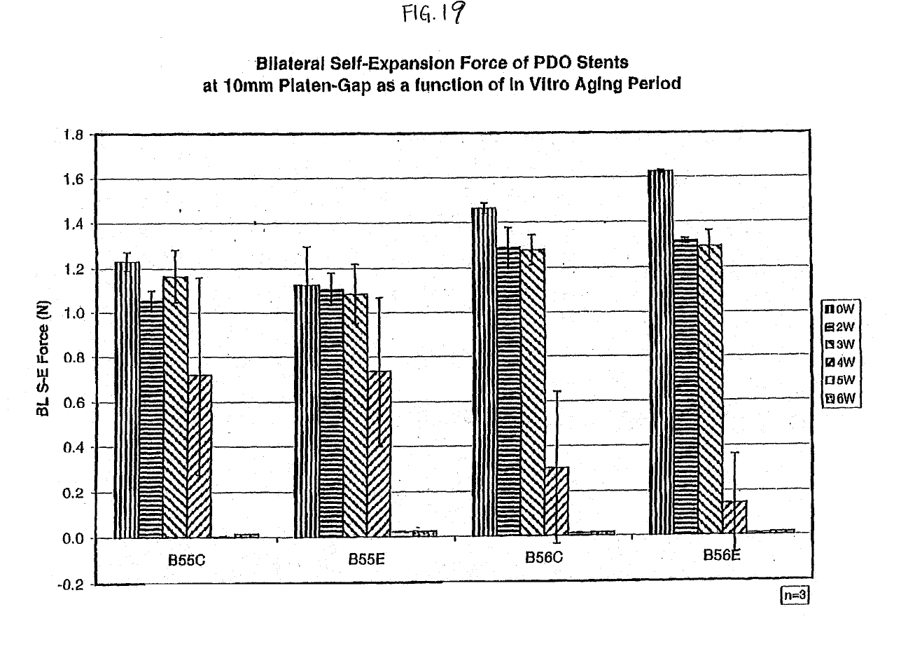

FIGS. 18 and 19 are bar charts that illustrate the compression resistance and self-expansion force as a function of in vitro aging for four bioresorbable fenestrated tube stents. The four test groups were subjected to different combinations of annealing and sterilization. FIGS. 18 and 19 show that all four test groups maintained approximately 80% to 95% of initial compression resistance and 88% to 100% of self-expansion force after three weeks of aging. Additionally, FIGS. 18 and 19 show that the annealed stents had approximately 18% to 23% higher initial compression resistance and approximately 25% to 45% higher initial self-expansion force than non-annealed stents. FIGS. 13 and 14 also show that ethylene oxide (eto) sterilization provides some slightly increased mechanical properties. The data as shown in FIGS. 18 and 19 illustrate bioresorbable stents 50 that have a functional life of approximately two to four weeks.

In yet another preferred embodiment a non-toxic radio-opaque marker is incorporated into the polymer blend prior to extruding the monofilaments used to weave the stent. Examples of suitable radio-opaque markers include, but are not limited to, Cage barium sulfate and bismuth trioxide in a concentration of between approximately 5% to 30%.

Table 1 represents the results obtained from testing different lots and configurations of the polymeric stents of the present invention. The stents polymers and were tested as described in Examples 1, 4 and 5.

TABLE 1

| | Raw Matl. | | MF NMR | | Gamma | | | | | |
|---|---|---|---|---|---|---|---|---|---|---|
| | Inh Visc | MF InhVisc | Monomer | Annealing | Treatment | | | | | |
| MF Batch | (dl/g) | (dl/g) | (%) | Conditions | (kGy) | 2W¹ | 3W | 4W | 5W | 6W |
| 537-05 | >8.0 | 2.22 | 3.68 | 90 C./4 hr | 50 | 59% | 34% | 24% | | 5% |
| 537-05 | >8.0 | 2.22 | 3.68 | 90 C./4 hr | 35 | 59% | 47% | 27% | | 6% |
| 537-03 | >8.0 | 1.96 | 1.1 | 90 C./4 hr | 50 | 95% | 93% | 84% | | 71% |
| 537-03 | >8.0 | 1.96 | 1.1 | 90 C./4 hr | 35 | 88% | 85% | 80% | | 69% |
| 537-02 | >8.0 | 2.28 | 1.31 | 90 C./4 hr | 50 | 90% | 87% | 83% | | 55% |
| 537-02 | >8.0 | 2.28 | 1.31 | 90 C./4 hr | 35 | 89% | 82% | 71% | | 17% |
| 537-01 | >8.0 | 1.93 | 1.23 | 90 C./4 hr | 50 | 92% | 83% | 78% | | 51% |
| 537-01 | >8.0 | 1.93 | 1.23 | 90 C./4 hr | 35 | 90% | 83% | 76% | | 58% |
| 537-07 | <4.5 | 2.26 | 0.81 | 90 C./4 hr | 35 | 96% | 93% | 92% | 88% | 81% |
| 537-07 | <4.5 | 2.26 | 0.81 | 90 C./4 hr | 35 | 104% | 93% | 88% | | 81% |
| 537-07 | <4.5 | 2.26 | 0.81 | 90 C./4 hr | 50 | 98% | 85% | 84% | 85% | 79% |
| 537-07 | <4.5 | 2.26 | 0.81 | 90 C./4 hr | 50 | 105% | 103% | 97% | | 90% |
| 537-06 | <4.5 | 1.95 | | 90 C./4 hr | 35 | 98% | 91% | 90% | 79% | 69% |
| 472-35 | >8.0 | 3.43 | 2.41 | 90 C./4 hr | 0 | 93% | 85% | 77% | 72% | 58% |
| 472-35 | >8.0 | 3.43 | 2.41 | 90 C./4 hr | 0 | 74% | 69% | 61% | 56% | 46% |
| 472-35 | >8.0 | 3.43 | 2.41 | 90 C./4 hr | 50 | 75% | 57% | 36% | 23% | 21% |
| 472-35 | >8.0 | 3.43 | 2.41 | 90 C./4 hr | 65 | 25% | 26% | 18% | 12% | 12% |
| 472-35 | >8.0 | 3.43 | 2.41 | 90 C./4 hr | 75 | 65% | 36% | 28% | 16% | 14% |
| 472-35 | >8.0 | 3.43 | 2.41 | 90 C./4 hr | 0 | 89% | 79% | 73% | 60% | 43% |
| 472-35 | >8.0 | 3.43 | 2.41 | 90 C./4 hr | 35 | 73% | 59% | 41% | 29% | 23% |
| 472-35 | >8.0 | 3.43 | 2.41 | 90 C./4 hr | 50 | 80% | 53% | 35% | 27% | 18% |
| 472-35 | >8.0 | 3.43 | 2.41 | 90 C./4 hr | 65 | 57% | 38% | 27% | 18% | 15% |
| 472-35 | >8.0 | 3.43 | 2.41 | 90 C./4 hr | 25 | 86% | 72% | 61% | 42% | 33% |
| 472-35 | >8.0 | 3.43 | 2.41 | 90 C./4 hr | 35 | 88% | 65% | 41% | 26% | 21% |
| 472-35 | >8.0 | 3.43 | 2.41 | 90 C./4 hr | 50 | 83% | 57% | 28% | 20% | 17% |
| 472-35 | >8.0 | 3.43 | 2.41 | 90 C./4 hr | 50 | 81% | | 48% | | |
| 472-35 | >8.0 | 3.43 | 2.41 | 90 C./4 hr | 65 | 73% | 43% | 19% | 14% | 10% |
| 472-35 | >8.0 | 3.43 | 2.41 | 90 C./4 hr | 65 | 87% | | 39% | | |
| 472-35 | >8.0 | 3.43 | 2.41 | 90 C./4 hr | 75 | 76% | 58% | 33% | 32% | 25% |
| 472-35 | >8.0 | 3.43 | 2.41 | 142 C./13 hr | 0 | 104% | 104% | 104% | 105% | 109% |
| 472-35 | >8.0 | 3.43 | 2.41 | 142 C./13 hr | 0 | 99% | 97% | 95% | 97% | 104% |
| 472-35 | >8.0 | 3.43 | 2.41 | 142 C./13 hr | 50 | 115% | 108% | 106% | 120% | 124% |
| 472-35 | >8.0 | 3.43 | 2.41 | 142 C./13 hr | 65 | 112% | 111% | 108% | 121% | 123% |
| 472-35 | >8.0 | 3.43 | 2.41 | 142 C./13 hr | 75 | 111% | 119% | 74% | 73% | 125% |
| 472-35 | >8.0 | 3.43 | 2.41 | 140 C./3 hr | 0 | 103% | 104% | 105% | 108% | 116% |
| 472-35 | >8.0 | 3.43 | 2.41 | 140 C./3 hr | 0 | 97% | 66% | 96% | 96% | 106% |
| 472-35 | >8.0 | 3.43 | 2.41 | 140 C./3 hr | 50 | 119% | 114% | 82% | 76% | 101% |
| 472-35 | >8.0 | 3.43 | 2.41 | 140 C./3 hr | 65 | 97% | 101% | 63% | 75% | 115% |
| 472-35 | >8.0 | 3.43 | 2.41 | 140 C./3 hr | 75 | 106% | 109% | 74% | 71% | 114% |

¹2W, 3W, 4W, 5W and 6W refer to the number of week strength is retained.

In closing, it is to be understood that the embodiments of the invention disclosed herein are illustrative of the principles of the present invention. Other modifications that may be employed are within the scope of the invention. Thus, by way of example, but not of limitation, alternative configurations of the bioresorbable, self-expanding stent may be utilized in the treatment of urethral stenoses. Accordingly, the present invention is not limited to that precisely as shown and described in the present invention.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitations of ranges of values herein are merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference.

What is claimed is:

1. A method for controlling a polymeric stent's in vivo functional life span comprising:
selecting a bioresorbable, biocompatible polymer composition,
determining a monomer content within said bioresorbable, biocompatible polymer composition,
adjusting said monomer content in said bioresorbable, biocompatible polymer to within a predetermined range.

2. The method according to claim 1 wherein said monomer content is adjusted in said bioresorbable, biocompatible polymer by the addition of monomer to said bioresorbable, biocompatible polymer composition prior to blending or extrusion.

3. The method according to claim 1 wherein said monomer content in said polymeric composition is adjusted prior to stent formation using a method selected from the group consisting of polymer extrusion pressure, temperature, residence time and combinations thereof.

4. The method according to claim 1 wherein said selected bioresorbable, biocompatible polymer composition has a high molecular weight (high inherent viscosity).

5. The method according to claim 1 wherein said polymeric biological stent is made from a method comprising weaving said polymeric stent from polymeric filaments or extruding a polymeric tube and cutting fenestrations into said polymeric tube.

6. A method for controlling bioresorbable stent in vivo functional life wherein said stent comprises a polymeric composition having monomer content within a predetermined range comprising:
adjusting said monomer content to within said predetermined range in said polymeric composition prior to stent formation using a method selected from the group consisting of polymer extrusion pressure and temperature, blending polymeric ingredients, having differing monomer content, adding monomer to said polymeric composition and combinations thereof.

7. The method according to claim 6 wherein said polymeric biological stent is made from a method comprising weaving said polymeric stent from polymeric filaments or extruding a polymeric tube and cutting fenestrations into said polymeric tube.

8. A method of producing a bioresorbable, polymeric stent comprising:
providing biocompatible, bioresorbable polymeric monofilaments wherein said polymeric monofilaments comprise a polymeric composition adjusted to have a monomer content within a predetermined range;
braiding said monofilaments into a latticed network, said latticed network having an alternating braiding pattern; and
annealing said latticed structure.

9. The method according to claim 8 wherein said monomer content in said polymer composition is adjusted prior to stent formation using a method selected from the group consisting of polymer extrusion pressure and temperature and residence time and combinations thereof.

10. The method according to claim 8 wherein said monomer content in said polymer composition is adjusted in said polymeric monofilament to within a predetermined range through a processes comprising blending polymeric ingredients having differing monomer contents.

11. The method according to claim 8 wherein said biocompatible, bioresorbable monofilaments are poly-L-lactide monofilaments.

12. The method according to claim 11 wherein said annealing step further includes heating said latticed structure to 90° C. in an inert atmosphere.

13. The method according to claim 12 wherein said inert atmosphere is selected from the group consisting of nitrogen, argon, and helium.

14. The method according to claim 12 wherein said inert atmosphere comprises a high vacuum.

15. The method according to claim 8 further comprising:
axially compressing said latticed structure by 30% to 60% prior to said annealing step.

16. The method according to claim 11 wherein said ratio of low molecular weight polymeric sub-units to high molecular weight polymeric molecules is adjusted in said polymer monofilament used to form said polymeric stent prior to stent formation using a method selected from the group consisting of polymer extrusion pressure and temperature, blending polymeric ingredients having differing monomer contents used to form said polymeric material used to form said polymeric biological stents and combinations thereof.

17. A method of producing a bioresorbable, polymeric stent comprising:
selecting a bioresorbable, biocompatible polymer composition,
determining a monomer content within said bioresorbable, biocompatible polymer composition,
adjusting said monomer content in said bioresorbable, biocompatible polymer to within a predetermined range,
extruding said polymer composition Into monofilaments,
braiding said monofilaments into a latticed structure, wherein said biocompatible, bioresorbable monofilaments are woven in an alternating braiding pattern; and
annealing said latticed structure in an inert atmosphere wherein said inert atmosphere is selected from the group consisting of nitrogen, argon, helium, and high vacuum.

18. The method according to claim 17 further comprising:
axially compressing said latticed structure on a mandrel by 30% to 60% prior to said annealing step.

19. The method according to claim 17 further comprising:
exposing said annealed latticed structure to gamma irradiation.

20. The method according to claim 19 wherein said latticed structure is exposed to approximately 35 kGy to 75 kGy total dose of gamma irradiation.

21. A method of producing a bioresorbable, self-expanding stent comprising:
(a) selecting a high molecular weight poly-L-lactic acid (PLLA) polymeric composition,
(b) determining a monomer content within PLLA;
(c) adjusting said monomer content in said PLLA to within a predetermined range;
(d) extruding said PLLA into monofilaments;
(e) braiding said poly-L-lactide monofilaments into a latticed structure, wherein said poly-L-lactide monofilaments are woven in an alternating under-two-over-two pattern;
(f) axially compressing said latticed structure on a mandrel by 30% to 60%
(g) annealing said latticed structure at approximately 90° C. for at least one hour in an inert atmosphere, wherein said inert atmosphere is selected from the group consisting of nitrogen, argon, helium, and high vacuum; and
(h) exposing said latticed structure to approximately 35 kGy to 75 kGy total dose of gamma irradiation.

22. A method of producing a stent comprising:
selecting a bioresorbable, biocompatible polymer composition,
determining a monomer content within said bioresorbable, biocompatible polymer composition,
adjusting said monomer content in said bioresorbable, biocompatible polymer to within a predetermined range,
forming a tubular sheath having fenestrations from said biocompatible, bioresorbable polymer; and
annealing said tubular sheath.

23. The method according to claim 22 wherein said forming step further comprises injection molding or extruding said tubular sheath.

24. The method according to 22 wherein said annealing step further comprises heating said tubular sheath for approximately one to three hours.

25. The method according to claim 22 wherein said annealing step further includes exposing said tubular sheath to an inert atmosphere inert atmosphere is selected from the group consisting of nitrogen, argon, and helium.

26. The method according to claim 24 wherein said annealing step further includes exposing said tubular sheath to a high vacuum.

27. The method according to claim 22 wherein said forming step further comprises laser cutting said fenestrations.

28. A method of producing a stent comprising:
selecting a bioresorbable, biocompatible polymer composition,
determining a monomer content within said bioresorbable, biocompatible polymer composition,
adjusting said monomer content in said bioresorbable, biocompatible polymer to within a predetermined range,
forming a tubular sheath from a biocompatible, bioresorbable polymer;
cutting fenestrations into said tubular sheath; and
annealing said tubular sheath for approximately one to three hours in an inert atmosphere.

29. The method according to claim 28 wherein said annealing step further includes exposing said tubular sheath to nitrogen, argon or helium.

30. The method according to claim 28 wherein said annealing step further includes exposing said tubular sheath to high vacuum.

31. A bioresorbable, self-expanding stent comprising:
a cylindrical sleeve having a first end and a second end;
a latticed network disposed between said first end and said second end of said cylindrical sleeve;
said latticed network formed from approximately forty monofilaments helically wound about a longitudinal axis of said cylindrical sleeve, wherein approximately twenty of said monofilaments are wound in a clockwise direction and approximately twenty said monofilaments are wound in a counter-clockwise direction, wherein said approximately forty monofilaments are braided in an alternating under-two-over-two braid pattern; and
said plurality of braided monofilaments comprises a PLLA composition wherein said PLLA composition has a monomer content adjusted such that said PLLA composition has a controllable in vivo lifetime.
